(19) United States
(12) Patent Application Publication
(10) Pub. No.: US 2013/0178472 A1
(43) Pub. Date: Jul. 11, 2013

(54) ARYLSULFONAMIDE PYRIDINE-PYRIDINONE DERIVATIVES, PREPARATION OF SAME, AND THERAPEUTIC USE THEREOF

(71) Applicants: Patrice BELLEVERGUE, Paris (FR); Patrick GRAILHE, Antony (FR); Gary MCCORT, Paris (FR); Stephen O'CONNOR, L'Oudon (FR); Olivier DUCLOS, Gentilly (FR)

(72) Inventors: Patrice BELLEVERGUE, Paris (FR); Patrick GRAILHE, Antony (FR); Gary MCCORT, Paris (FR); Stephen O'CONNOR, L'Oudon (FR); Olivier DUCLOS, Gentilly (FR)

(73) Assignee: SANOFI, Paris (FR)

(21) Appl. No.: 13/539,706

(22) Filed: Jul. 2, 2012

Related U.S. Application Data
(63) Continuation of application No. PCT/FR2011/050018, filed on Jan. 6, 2011.

(30) Foreign Application Priority Data
Jan. 7, 2010 (FR) 1050081

(51) Int. Cl. C07D 471/04; A61K 31/4375; C07F 5/02; A61K 45/06; A61K 31/444; A61K 31/5377

(52) U.S. Cl. CPC C07D 471/04; A61K 31/444; A61K 31/5377; C07F 5/025; A61K 45/06; A61K 31/4375
USPC 514/234.5; 514/300; 546/123; 544/127; 546/13

(57) ABSTRACT

The invention relates to pyridine-pyridinone derivatives general formula (I):

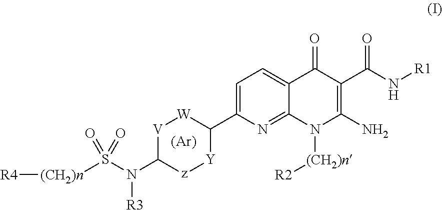

in which R1, R2, R3, R4, n, n′, V, W, Y, Z, Ar are as defined in the description, and to their methods of preparation and their therapeutic applications.

This application is a Continuation of International Application No. PCT/FR2011/050018, filed Jan. 6, 2011, which claims priority from French Application No. 1050081, filed on Jan. 7, 2010, both of which are incorporated herein by reference in their entireties.

The present invention relates to pyridino-pyridinone derivatives substituted at the 7-position with an arylsulfonamide, to their preparation and to their therapeutic application as inhibitors of protein kinases such as p70S6 (S6K1) and/or of PDGFR-TK (platelet derived growth factors), or of other kinases.

1) Protein Kinase p70S6K:

### I. p70 S6Kinase or S6K1: Overview, Structure, Activation

The ribosomal p70 S6 Kinase (S6K1, formerly p70S6K) is a serine/threonine kinase (of the AGC kinase family) of the PI3-kinase/mTOR pathway among the first described as activated by insulin and many growth factors. This kinase participates in the regulation of two cellular processes: protein synthesis and cell growth (proliferation and size of the cells), via its main substrate, the ribosomal protein S6 of the 40S subunit. (Avruch J. 2001). Cloned in 1991 by Grove et al., two isoforms, resulting from an alternative splicing of the mRNA, encode 2 protein sequences: p85 S6K (α-I, 525 amino acids) and p70S6K (α-II, 502 amino acids) have been identified. The latter isoform is mainly located in the cytosol while the α-I isoform is nuclear (presence of a nuclear localization site on the N-terminal extension of 23 amino acids). S6K1 is expressed ubiquitously.

S6K1 exhibits 70% amino acid homology with S6K2 (formerly p70 beta S6 kinase), also activated by mTOR, in which 7 phosphorylation sites (serine or threonine) are conserved.

The structure of S6K1 comprises four modules: a noncatalytic domain at the N-terminal end (I), a central catalytic domain (II), an extension of the kinase domain (III) and finally an auto-inhibitory domain at the C-terminal end (IV). The activation of this kinase requires sequential phosphorylation in 4 stages of serine or threonine sites located on various domains which will modify its overall conformation, allowing it to acquire its enzyme activity (Pollen N. 1997, Dennis JBC1998).

### II. S6K1 in the PI3K/mTOR Signaling Pathway

The upstream signaling of S6K1 results from the activation of many membrane G Protein Coupled Receptors (GPCRs), which control cell growth, proliferation and differentiation. After binding of ligands such as growth factors (for example PDGF, EGF), nutrients or hormones (for example amino acids, glucose or insulin), the activation of their receptors results in the recruitment of PI3-Kinase, triggering a phosphorylation cascade via PDK1 which phosphorylates Akt, activating mTOR (via TSC1/2 and Rheb) which finally activates S6K1, one of the two main effectors of mTOR. Finally, the pro-apoptotic protein BAD is phosphorylated at S136 by S6K1 which inactivates and improves cell survival (Harada et al. PNAS 2001).

More recently, chaperonin containing TCP1, CCT, was reported as substrate for S6K1 and plays a role in the folding of neosynthesized proteins such as actin, tubulin and several cell cycle proteins, also suggesting a role for S6K1 in cell cycle regulation (Abe et al. JBC2009).

### III. Applications of S6K1 Inhibitors

By virtue of its regulatory activity on cell growth and protein synthesis, S6K1 is involved in many physiopathological processes. S6K1 inhibitors can therefore find applications in many therapeutic domains: cardiovascular diseases such as heart failure following myocardial hypertrophy, atherosclerosis and restenosis following excessive proliferation of the smooth muscle cells of the arteries or kidney failure. Metabolic disorders and in particular diabetes and obesity represent other possible therapeutic applications for S6K1 inhibitors. Fibrotic diseases, such as hepatic, pancreatic, pulmonary, cardiac and perivascular fibrosis, resulting from excessive synthesis of extracellular matrix and excessive proliferation of fibroblasts, stellar cells or smooth muscle cells, regulated inter alia by the activity of S6K1, also constitute therapeutic indications for these inhibitors. Finally, any tumors with deregulations of the PI3K/Akt/mTOR pathway could benefit from treatment with S6K1 inhibitors.

### IV. Role of S6K1 in the Cardiovascular System

Hypertrophy of the cardiomyocytes due to an excessive protein synthesis is one of the key mechanisms involved in the development of myocardial hypertrophy which results in heart failure. The mTOR/S6K1 signaling pathway is one of the main systems for regulating cell growth by regulating protein synthesis and cell proliferation. Numerous studies in vivo have shown the therapeutic potential of inhibitors of this pathway, including rapamycin, inhibitor of mTOR (mTORC1 complex) which blocks the activation of S6K1. Rapamycin reduces cardiac hypertrophy following a cardiac overload by constriction of the aorta in mice and rats (Gao et al. Hypertension 2006, Boluyt M. et al. Cardiovasc. Drug Therap. 2004, Shioi et al. Circulation 2003). Rapamycin reduces the hypertrophy of the left ventricle, preserves the contractile function and reduces cardiac fibrosis (reduction of collagen) by a mechanism involving the mTOR/S6K1 pathway since the phosphorylation of the ribosomal protein S6 and eIF4E is inhibited (Gao J Hypertension 2006).

The involvement of the mTOR/S6K1 pathway in the hyperplasia of the smooth muscle cells of the artery is demonstrated by the inhibitory role of rapamycin in the growth of the smooth muscle cells of the artery in vitro and has been used in the prevention of restenosis of the coronary artery after transluminal angioplasty using stents coated with rapamycin (Moses et al. N Engl. J. Med. 2003) or after systemic injection (ORAR Trial, Rodriguez et al. J. Invasive cardiol. 2003). In particular, in diabetic patients, a clinical study has shown that stents impregnated with rapamycin significantly reduce the risks of restenosis after coronary angioplasty (SIRIUS Substudy, Moussa et al. Circulation 2004). The compounds of the present invention could therefore have an application in the prevention of restenosis and atherosclerosis.

### V. Role of S6K1 in the Fibrosis Process

Excessive tissue repair following chronic lesions/stimuli resulting in an excessive synthesis of extracellular matrix and excessive differentiation of the fibroblasts into myofibroblasts characterizes the fibrosis process which occurs in numerous tissues. By virtue of its regulatory activity on protein synthesis and cell growth, S6K1 is highly involved in fibrosis; the inhibitors of the present invention may therefore find applications in fibrosis of the liver, the pancreas, the skin, the lung, the heart or the kidney.

The role of S6K1 in liver fibrosis and in particular in the process of activation of hepatic stellar cells (for a review cf Parsons J. Gastro. Hepatol. 2007) has been demonstrated in experiments in vivo in the liver fibrosis model in rats by ligation of the bile duct or the inhibition of mTOR by rapamycin reduces the activation of S6K1, reduces fibrosis and improves portal hypertension, a functional effect accompanied by a reduction in the mRNA of TGFβ, CTGF, PDGFβ as well as a reduction in the phosphorylated S6K1 (Biecker et al. JPET 2005). In another model of fibrosis of the liver induced in rats by carbon tetrachloride, rapamycin reduces collagen deposits and transglutaminase activity in vivo and completely blocks the proliferation of stellar cells which is induced by PDGFβ (Zhu et al. Gastroenterology 1999). In 2 models of liver fibrosis induced by ligation of the bile duct (BDL) or by injection of dimethylnitrosamine (DMN), a kinetic study ex vivo of the activity of ERK1/2 and of S6K1 has shown that the activity of this kinase precedes the activation and proliferation of the hepatic stellar cells, the S6K1 activity peak being 6 hours in the DMN model and 72 hours in the BDL model (Svegliati-Baroni et al. J. Hepatol. 2003). In vitro, the activation of stellar cells by PDGFb and IGF-1 involves S6K1 and rapamycin inhibits the proliferation of the stellar cells and the activation of S6K1 (Bridle et al. JLCM 2006). This inhibitor has also proved capable of blocking the overexpression of MMPI3, of collagen I and the activation of S6K1 in stellar cells activated by TGFβ (Lechuga et al. J. AJPGLP 2004).

At the level of skin fibrosis, a high expression of S6K1 has been demonstrated in keloid scars. Rapamycin reduces collagen, fibronectin, actin α (α-SMA) (Ong et al. Exp. Dermatol. 2007).

At the level of lung fibrosis, rapamycin prevents the initiation and progression of lung fibrosis in a transgenic mouse model overexpressing TGFα in the lung. Furthermore, this inhibitor blocks the phosphorylation of S6K1 induced by TGFα and the depositions of collagen in the lung (Korfhagen et al. Am. J. resp. Cell Mol. Biol. 2009).

### VI. Oncology:

Inhibitors of P70S6K have applications in oncology, in particular in:
breast cancer, where the amplified and overexpressed S6K1 gene is present in 7.5-10.2% primary breast cancers (Barlund et al. 2000, Wu et al. 2000, Couch et al. 1999); this overexpression is associated with a poor prognosis independent of the amplification of HER2 (Barlund et al. 2000).
liver cancer (hepatocarcinoma, HCC): overexpression of S6K1 reported by Sahin et al (2004).
glioblastoma (Riemenschneider et al. 2006)
thyroid cancer (Miyakawa et al. 2003)
ovarian and cervical cancers (Wong et al. 2000). S6K1 promotes the process of transition of epithelial cells to mesenchymal cells (EMT) in ovarian cancer cells (Pon et al. Cancer Res. 2008).

### 2) PDGF-R Receptor Tyrosine Kinases:

The PDGF-R receptors are members of the class III family of receptor tyrosine kinases (RTK). The binding of ligands to RTKs induces dimerization of the receptors, the activation of their tyrosine kinase portion which leads to the transphosphorylation of the tyrosine residues (Weiss & Schlessinger, 1998).

The binding of ligands to these RTKs induces dimerization of the receptors, the activation of their tyrosine kinase portion which leads to the transphosphorylation of the tyrosine residues (Weiss & Schlessinger, 1998). These phosphorylated residues thus serve as anchoring point for the intracellular signaling proteins which in fine cause various cellular responses: maintenance, division, proliferation, differentiation, or else cell migration. (Claesson-Welsh, 1994).

Two isoforms of PDGF receptors have been identified, the PDGF-Ralpha chain and the PDGF-Rbeta chain, which, following the attachment of their ligands, homo- or heterodimerize and induce intracellular signaling. The PDGF receptors are mainly expressed by cells of mesenchymal origin and are found in particular on the fibroblasts, the smooth muscle cells, the pericytes and the glial cells (Ross et al., 1986, Heldin, 1992).

“Platelet Derived Growth Factor”, PDGF, a protein having a molecular weight of about 30,000 daltons, is mainly secreted by the platelets, secondarily by the endothelium, the vascular smooth muscles and the monocytes. It is formed of two polypeptide chains linked to each other by disulfide bonds forming either homodimers or heterodimers. Four genes (7p22, 22q13, 4q31 and 11q22) have been described as encoding 4 different polypeptide chains (A, B C and D), which once dimerized give five biologically active ligands PDGF-AA, BB, CC, DD and AB (for a review, Yu et al, 2003). A binding specificity exists, including in particular PDGF-AA for the alpha isoform of the receptor, PDGF-D for the BB form, and PDGF-C for the alpha and alpha/beta forms. The PDGF ligands are potent mitogens, but are also involved in the phenomena of cell migration, survival, apoptosis and transformation.

The inhibitors of the PDGF-R alpha, beta function are involved in various therapeutic fields. Among the physiopathological phenomena in which these receptors may be involved are cancers with or without metastases targeting tumor cells and/or (vascular, fibroblast) cells of the tumor environment, fibroses and vascular diseases:

Advantageously, AML (acute myeloid leukemia)-type blast cells can also overexpress other receptors with kinase activity such as c-kit or else PDGF-R.

### Myeloproliferative/Dysplastic Syndromes

Quite frequently, cytogenetic abnormalities following chromosomal translocations have been reported in myeloproliferative syndromes. These rearrangements generate deregulated fusion proteins with tyrosine kinase activity which are involved in the proliferation of myeloid blast cells.

Fusion Proteins with PDGF-R Beta Kinase Activity

Fusion proteins with PDGF-R beta kinase activity consist of the intracellular portion of PDGF-R-beta and, on the other hand, of an N-ter domain of another protein (in general a transcription factor). The following have been reported in particular in chronic myelomonocytic leukemias (CMML): RabS/PDGF-Rbeta, H4-PDGF-Rbeta, HIP1-PDGF-RB or else Tel/PDGF-R beta. The latter is the most widely represented. It is derived from the translocation t(5; 12)(q31; p12) and encodes a fusion protein consisting of the N-terminal part of the transcription factor Tel and of the C-terminal part of PDGF-Rbeta. An oligomerization domain present in the Tel part leads to a dimerized form of the fusion protein and to the constitutive activity of the kinase domain. This protein has been shown in vitro to be capable of transforming hematopoietic cells on several occasions and in particular in detail in the article by M. Carrol et al., (PNAS, 1996, 93, 14845-14850). In vivo, this fusion protein leads to a myeloid cell hyperproliferation (Ritchie et al., 1999).

Furthermore, in animals and in the clinical setting in humans, it has been shown that inhibitors of tyrosine kinase activity inhibit the proliferation of blast cells and make it possible to check the leukemogenesis process.

Fusion Proteins with PDGF-R Alpha Kinase Activity

Two fusion proteins involving PDGF-R alpha have been reported: bcr-PDGF-Ralpha which is present in an atypical chronic myeloid leukemia (CML) and FIP1L1-PDGF-Ralpha which is found in a subpopulation of leukemias, CELs “eosinophilic leukemias”, derived from a hypereosinophilic syndrome (Griffin et al., 2003). This fusion protein bears a constitutive activity of the kinase domain of PDGF-R alpha and is responsible for the anarchic proliferation of these cells.

Inhibitors of the kinase activity of PDGF-R alpha have shown efficacy on the proliferation of positive FIP1L1-PDGF-R alpha cells and recently an inhibitory compound got the indication for HES/CEL.

Thus, inhibiting the kinase activity of PDGF-Ralpha and beta as the compounds of the invention do has proved to be of therapeutic interest for AMLs.

### A. Solid Cancers

Inhibitors of the tyrosine kinase activity of PDGF-R alpha and beta receptors may be of interest for solid cancers either by directly targeting the tumor cell which by autocriny or paracriny is sensitive to the PDGF-R TK inhibiting activity, or by targeting the cells of the environment by destabilizing the network in order to promote the association with other therapeutic agents. Examples of solid cancers are Ewing's sarcoma, gastrointestinal stromal tumors (GIST), dermatofibrosarcomas, gliomas, glyoblastomas, hemangiomas as well as desmoid tumors. The compounds of the invention are of interest for the treatment of such solid cancers.

### B. Targeting PDGF-RTK in the Tumor Environment

### Angiogenesis

The cells of the tumor environment form an integral part of the development of the cancer whether in the case of a primary or secondary tumor (metastases). Among the cells of the environment which express PDGF-R and for which the role of this receptor has been demonstrated are the mural cells of the vessels, that is to say the pericytes and the smooth muscle cells but also the activated fibroblasts.

Angiogenesis is a process for generating new capillary vessels from pre-existing vessels or by mobilization and differentiation of bone marrow cells. Thus, both uncontrolled proliferation of the endothelial cells and a mobilization of angioblasts from the bone marrow are observed in the tumor neovascularization process. It has been shown in vitro and in vivo that several growth factors stimulate endothelial proliferation such as VEGF and FGFs. In addition to these mechanisms, it has also been demonstrated that the mural cells such as the pericytes and the smooth muscle cells participate in the stabilization of the newly-formed vessels. The invalidation of PDGF-R beta causes a deficiency in the pericytes in mice and leads to the death of the animals at the end of gestation due to microhemorrhages and edemas (Hellström et al, 1999, Hellström et al, 2001). In an elegant study of transplantation, the expression of PDGF-R-beta by the pericytes has been shown to be necessary for their recruitment at the level of the tumor vessels via the retention of PDGF-B by the endothelial cells but also by the PDGF-B secreted by the tumor cells (Abramsson et al, 2003). In the Rip1Tag2 transgenic model of pancreatic tumor, Song et al. have also shown the expression of PDGF-R beta on the perivascular progenitors in the marrow derived from bone marrow, progenitors which differentiate into mature pericytes around the tumor.

The importance of blocking the activity of PDGF-R on the tumor pericytes has been demonstrated by the use of an inhibitor of the tyrosine kinase activity of PDGF-R in animal models (transgenic model of pancreatic tumor and implantation of glioma tumor), and the effect on tumor growth turns out to be profound in combination with an inhibitor of the kinase activity of VEGF-R (Bergers et al., 2003). Literature data (Cao et al, 2002, Fons et al., 2004) have demonstrated the intervention of PDGF-R alpha and PDGF-C in angiogenesis and in the differentiation of the endothelial progenitors into cells of the pericyte type and smooth muscle cells.

In the light of these various studies, it is apparent that the compounds of the invention are of interest for the treatment of solid cancers by their effect on the cells of the environment and this being in combination with other therapeutic agents such as cytotoxic agents or inhibitors of angiogenesis.

### Activated Fibroblasts

PDGF-R is abundant in the tumor stroma and is found on the activated fibroblasts (myofibroblasts). It has been shown in two studies that the combination of inhibitors or antagonists of PDGF-R with cytotoxic agents leads to a reduction in the microdensity of the vessels in ovarian cancers (Apte et al., 2004) and in pancreatic cancers (Hwang et al., 2003). PDGF-R beta regulates the pressure of the interstitial tissue of the tumor (Heuchel et al., 1999) and the co-administration of inhibitors of PDGF-R and chemotherapeutic agents improves their delivery in tumor cells by reducing the intratumor pressure (Griffon-Etienne, 1999). Finally, in a murine model, the administration of an inhibitor of the kinase activity of PDGF-R improves the consumption of chemotherapeutic agents by the tumor and thus increases their efficacy (Griffon-Etienne, 1999; Pietras et al., 2002; Pietras et al., 2003). The activated fibroblasts present in the tumor stroma therefore represent a novel therapeutic target in oncology (for a review see Bouzin & Feron, 2007).

### Metastases

Several studies show that the PDGF-R and PDGF-ligand pair is involved in the development of metastases, certainly by their action on angiogenesis and metastatization by the blood circulation, but also by a direct effect on lymphangiogenesis and therefore the metastases disseminated by the lymphatic vessels. One review documents in particular the direct role of PDGF-BB in lymphangiogenesis and lymphatic metastases (Cao et al., 2005). However, the majority of the studies involve the expression of PDGF-R in the environment of the metastases which promote the establishment and development of secondary tumors. The example most frequently reported is the development of bone metastases, of prostate cancer.

In the light of these various studies, it is apparent that the compounds of the invention are of interest for the treatment of solid cancers by their effect on the cells of the environment and this being in combination with other therapeutic agents such as cytotoxic agents or inhibitors of angiogenesis.

### C. Fibroses

Fibroses are often the cause of a primary event such as a cancer, radiotherapy treatment, hepatitis, alcoholemia. The implication of PDGF is clearly demonstrated in pulmonary fibrosis (including asbestosis), renal fibrosis (glomerulonephritis), medullar fibrosis (often associated with megakaryocytic leukemias), induced by radiotherapy as well as hepatic and pancreatic fibroses (linked to alcoholemia or to hepatitis) (for a review see JC Bonner, 2004). An overexpression of PDGF has been in particular clearly shown and results in in vivo models with inhibitors of the PDGF-R TK activity have also been reported. Among these studies, that of Einter et al., (2002) has shown that PDGF-CC is a potent inducer of renal fibrosis. The authors tested the efficacy of a neutralizing antibody in a model of unilateral urethra ligation, where fibrosis develops particularly rapidly. They observed a very marked antifibrosing effect with a reduction in the accumulation of myofibroblasts, a reduction in the accumulation of extracellular matrix and a reduction in collagen IV deposits. Another study carried out in a model of pulmonary fibrosis induced by bleomycin in mice has shown the efficacy of an inhibitor of the TK activity of PDGF-R on the prevention of fibrosis by inhibition of the proliferation of mesenchymal cells (Aono et al., 2005). In a model of fibrosis induced by asbestos, a PDGF-R TK inhibitor reduced the progression of fibrosis in the pulmonary parenchyma and the deposition of collagen (Vuorinen K, Gao F, Oury T D, Kinnula V L, Myllarniemi M. Imatinib mesylate inhibits fibrogenesis in asbestos-induced interstitial pneumonia. Exp Lung Res. 2007 September; 33(7): 357-73). Several teams have shown the involvement of PDGF-R in hepatic fibrosis. It has been clearly shown that PDGFBB and DD possess profibrogenic characteristics on hepatic stellate cells (Rovida et al., 2008; Borkham-Kamphorst et al., 2007). In vivo, a PDGF-R TK inhibitor is capable of reducing early fibrogenesis in a model of bile duct ligation in rats (Neef et al., 2006).

Accordingly, in the light of the literature data, the compounds of the invention appear to be of therapeutic interest for various types of fibrosis.

### D. Vascular Diseases: Atherosclerosis & Restenosis, Arteriosclerosis

The proliferation and migration of vascular smooth muscle cells contribute to intimal hypertrophy of the arteries and thus plays a major role in atherosclerosis and in restenosis following angioplasty and endoarterectomy. It has been clearly demonstrated in vitro and in vivo in animal models that PDGF is involved in these phenomena. In vivo, an increase in the expression of PDGF in a “vein graft” model in pigs has been shown in particular. Furthermore, it has also been shown that an inhibitor of the TK activity of PDGF-R substantially reduced the size of the lesions of the thoracic and abdominal artery in diabetic mice ApoE-KO (animals treated with streptozotocin). Another study has shown that the inhibition of the signaling induced by PDGF (antisense TK or PDGF A) leads to a reduction in the formation of the neointima in “balloon injury” and “coronary artery restenosis” models. (Deguchi J, 1999, Ferns et al., 1991, Sirois et al, 1997, Lindner et al., 1995).

Thus, inhibitors of the tyrosine kinase activity of PDGF-R, such as the compounds of the present invention, represent a therapy of choice, either alone, or in combination with compounds that are antagonists of other growth factors involved in these pathologies such as FGF, in the treatment of pathologies linked to the proliferation of vascular smooth muscle cells such as atherosclerosis, restenosis post-angioplasty or following the fitting of endovascular prostheses (stents) or during aortocoronary bypass.

The compounds of the invention, by virtue of their inhibitory activity on the TK activity of PDGF-R, have proved advantageous for treating these vascular diseases.

### E. Others

Other pathologies appear to be possible indications for the compounds of the invention including idiopathic pulmonary arterial hypertension (PAH). PAH characterized by a high and continuous increase in pressure in the pulmonary artery leads to right ventricular failure and often the death of the patient. It is associated with the increase in the proliferation and migration of the smooth muscle cells of the pulmonary vessels. Schermuly et al. (2005) have shown that the inhibition of the tyrosine kinase activity of the PDGF receptors considerably improves the progression of the disease. For that, they used inter alia an experimental pulmonary arterial hypertension model in rats, obtained by the administration of monocrotaline for 28 days. All the treated rats survived whereas 50% of them died in the untreated control group.

The subject of the present invention is compounds corresponding to the formula (I):

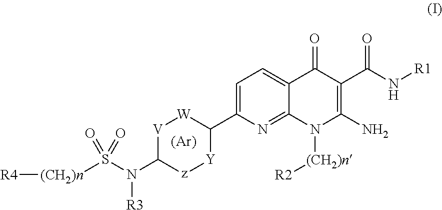

in which
n represents 0, 1, 2 or 3;
n′ represents 0, 1, 2, 3 or 4;
R1 represents an alkyl group;
R2 represents:
   (i) a cycloalkyl group,
   (ii) an alkyl group, or
   (iii) an alkoxy group,
   said cycloalkyl, alkyl or alkoxy groups being optionally substituted with one or more halogen atoms;
R3 represents:
   (i) a hydrogen atom, or
   (ii) a —C(O)alkyl group;
Ar represents a 5- or 6-membered aryl or heteroaryl ring in which Y, Z, V and W:
   (a) represent, independently of each other,
      (i) a ═CH— group,
      (ii) a ═C(R5)-group in which R5 represents:
         an alkyl group,
         a halogen atom, or
         an alkoxy group,
      (iii) a heteroatom chosen from the nitrogen atom, the sulfur atom and the oxygen atom,
   (b) at most one among Y, Z, V and W being optionally absent, it being understood that, when Ar represents a heteroaryl chosen from pyrrolyl, imidazolyl, pyrazolyl and triazolyls, at least one of the nitrogen atoms of said heteroaryl may be optionally substituted with a group R6 chosen from an alkyl group,
R4 represents a group chosen from:
   an alkyl group;
   an alkoxyalkyl group,
   a group —NRR′ with R and R′, which may be identical or different, representing, independently of each other, a hydrogen atom, an alkyl group or a —(C3-C6)cycloalkyl group,
   a cycloalkyl group,
   an alkenyl group,
   an aryl group, said aryl groups being optionally substituted with at least one halogen atom, and/or with at least one group chosen from a —(C1-C5)alkyl, haloalkyl, nitrile, haloalkyloxy, alkoxy, nitro group and the groups —NRR′ with R and R′, which may be identical or different, representing, independently of each other, a hydrogen atom or a group chosen from alkyl groups and —(C3-C6)cycloalkyl groups,
   a heteroaryl group, said groups comprising at least one heteroatom chosen from the nitrogen or sulfur atom, said heteroaryl groups being optionally substituted with at least one group chosen from alkyl groups and a heterocycloalkyl group comprising at least one heteroatom chosen from the nitrogen and oxygen atoms;
      it being understood that, when a heteroaryl group is chosen from pyrrolyl, imidazolyl, pyrazolyl and triazolyls, at least one of the nitrogen atoms of said heteroaryl may be substituted with a group R6 chosen from an alkyl group,
   a heterocycloalkyl group comprising at least one heteroatom chosen from the nitrogen, sulfur and oxygen atoms and being optionally substituted with at least one substituent chosen from (i) halogen atoms, (ii) haloalkyl groups, (iii) alkyl groups, advantageously linear or branched —(C1-C4)alkyl groups, and (iv) cycloalkyl groups,
      it being understood that when the heterocycloalkyl groups are chosen from pyrrolinyl, pyrrolidinyl, imidazolidinyl, imidazolinyl, pyrazolinyl, pyrazolidinyl, piperidinyl, morpholinyl, piperazinyl and thiomorpholinyl, at least one of the nitrogen atoms of said heterocycloalkyl may be optionally substituted with a group R6 chosen from an alkyl group,
         in the form of an acid, a base or an addition salt with an acid or a base.

The compounds of formula (I) may contain one or more asymmetric carbon atoms. They may therefore exist in the form of enantiomers or diastereoisomers. These enantiomers, diastereoisomers, and mixtures thereof, including racemic mixtures, form part of the invention.

For example, when R4 represents a heterocycle, the absolute configuration of a carbon substituted on said heterocycle may be R or S.

The compounds of formula (I) may exist in the form of bases or addition salts with acids. Such addition salts form part of the invention.

These salts may be prepared with pharmaceutically acceptable acids, but the salts of other acids useful, for example, for the purification or isolation of the compounds of formula (I), also form part of the invention.

The compounds of formula (I) may also exist in the form of solvates or hydrates, namely in the form of associations or combinations with one or more molecules of solvent or water, in crystalline or amorphous form. Such solvates and hydrates also form part of the invention.

The subject of the invention is also a method for preparing a compound of formula (I) according to the invention, characterized in that a compound of formula (IXa):

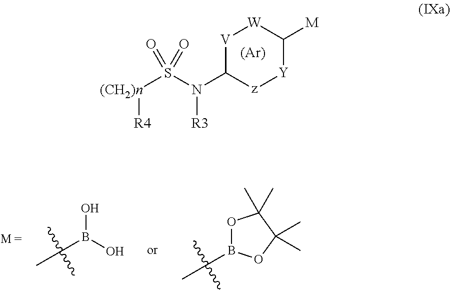

is reacted with a compound of formula (VII), in the presence of a coupling catalyst and a base as defined below,

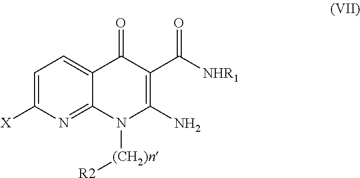

where R1, R2, R3, R4, n, n′, V, W, Y, Z and Ar are as defined above, X represents a leaving group defined below, advantageously X represents a halogen, more advantageously still X represents a chlorine atom and M is as defined above.

According to another aspect, the subject of the invention is also a method for preparing a compound of formula (I) according to the invention, characterized in that a compound of formula (IXb)

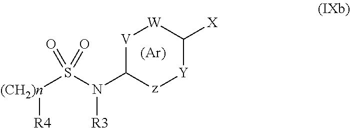

is reacted with a compound of formula (VIII),

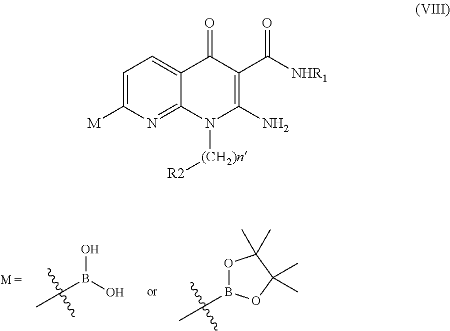

where R1, R2, R3, R4, n, n′, V, W, Y, Z and Ar are as defined above, X represents a leaving group defined below, advantageously X represents a halogen, more advantageously still X represents a bromine or iodine atom and M is as defined above.

In the context of the present invention, unless otherwise stated in the text, there is understood by:
a halogen atom: a fluorine, chlorine, bromine or iodine atom;
a heteroatom: a nitrogen, oxygen or sulfur atom;
an alkyl group: a linear or branched saturated aliphatic group which may contain 1, 2, 3, 4, 5 or 6 carbon atoms (abbreviated —(C1-C6)alkyl). Advantageously, this is a —(C1-C4)alkyl group. By way of examples, there may be mentioned as (i) —C1alkyl group, the methyl group, as (ii) —C2alkyl group, the ethyl group, as (iii)-C3alkyl group, the propyl, isopropyl group, as (iv) —C4alkyl group, the butyl, isobutyl, tert-butyl group, as (v) —C5alkyl group, the pentyl, isopentyl group, as (vi) —C6alkyl group, the hexyl group;
an alkylene group: a linear or branched saturated divalent alkyl group as previously defined, which may contain 1, 2, 3, 4, 5 or 6 carbon atoms (abbreviated —(C1-C6)alkylene). By way of example, there may be mentioned the methylene (or —CH₂—), ethylene (or —CH₂—CH₂—), propylene (—CH₂—CH₂—CH₂—) radicals;
an alkenyl group: an aliphatic group comprising at least 2 carbon atoms and being mono- or polyunsaturated. Advantageously, this is a C2-C10 group comprising at least one C═C double bond, more advantageously still a C2-C6 group comprising at least one C═C double bond;
a cycloalkyl group: a cyclic alkyl group which may contain 3, 4, 5, 6, 7, 8, 9 or 10 carbon atoms, also abbreviated —(C3-C10)cycloalkyl. Advantageously, this is a —(C3-C5)cycloalkyl group. By way of examples, there may be mentioned the cyclopropyl, methylcyclopropyl, cyclobutyl, cyclopentyl, cyclohexyl, cycloheptyl, adamantyl and pentalene groups;
an alkoxy or alkyloxy group: an —O-alkyl radical where the alkyl group is as previously defined. By way of examples, there may be mentioned the —O—(C1-C5)alkyl or —(C1-C5)alkoxy groups, and in particular as (i) —O—C1 alkyl group, the —Omethyl group, as (ii) —O—C2alkyl group, the —Oethyl group, as (iii) —O—C3alkyl group, the —Opropyl, —Oisopropyl group, as (iv) —O—C4alkyl group, the —Obutyl, —Oisobutyl, —Otert-butyl group, as (v) —O—C5alkyl group, the —Opentyl, —Oisopentyl group;
an alkoxyalkyl group: a radical of formula -alkylene-O-alkyl, where the alkyl and alkylene groups, comprising the same number of carbons or not comprising the same number of carbons, are as previously defined. By way of examples, there may be mentioned the groups —(C1-C6)alkylene-O—(C1-C6)alkyl, with —(C1-C6)alkylene- and —(C1-C6)alkyl as defined above;
a haloalkyl group: an alkyl group as defined above substituted with 1, 2, 3, 4 or 5 halogen atoms, as previously defined. There may be mentioned for example the groups -halo(C1-C5)alkyl, with (C1-C5)alkyl as defined above, and in particular the trifluoromethyl group (abbreviated —CF₃);
a haloalkyloxy group: a haloalkyl-O— group where the haloalkyl group is as defined above;
an aryl group: a cyclic aromatic group comprising 6, 7, 8, 9 or 10 carbon atoms.

By way of examples of aryl groups, there may be mentioned the phenyl group (abbreviated Ph) or a naphthyl group;
an arylalkyl group: an aryl group, as defined above, substituted with at least one alkyl group, as defined above. Advantageously, this refers to -alkyl-aryl radicals.

There may be mentioned, for example, benzyl, that is to say the —CH₂-Ph radical;
an aryloxy group: a radical of formula —O-aryl, where the aryl group is as previously defined;
a heteroaryl group: a cyclic aromatic group comprising 2, 3, 4 or 5 carbon atoms and comprising 1 to 3 heteroatoms, which may be chosen from the nitrogen atom, the oxygen atom and the sulfur atom, independently of each other, so as to be identical or different, when they are 2 in number or independently of each other, so as to be identical or different, when they are 3 in number. There may be mentioned, for example, the pyridinyl, pyrrolyl, pyrazolyl, furanyl, pyrazinyl, pyrimidyl, imidazolyl, thiophenyl, thiazolyl, 1,2,3-triazolyl and 1,2,4-triazolyl groups;
a heterocycloalkyl: an optionally bridged cyclic alkyl group comprising 5, 6 or 7 carbon atoms and comprising 1, 2 or 3 heteroatoms which may be chosen, independently of each other, so as to be identical or different, when they are 2 in number or independently of each other, so as to be identical or different, when they are 3 in number, from the nitrogen atom, the oxygen atom or the sulfur atom. There may be mentioned in particular piperidinyl, piperazinyl, pyrrolidinyl, hexamethyleneimino, tetrahydrofuranyl, morpholinyl, 1,1-dioxydotetrahydrothienyl groups;
a protecting group Pg: a group which makes it possible, on the one hand, to protect a reactive functional group such as a hydroxyl or an amine during a synthesis and, on the other hand, to regenerate the reactive functional group intact at the end of the synthesis. Examples of protecting groups and methods of protection and deprotection are given in “Protective Groups in Organic Synthesis”, Green et al., 2^{nd} Edition (John Wiley & Sons, Inc., New York), 1991;
a leaving group: a group which may be readily cleaved from a molecule by breaking a heterolytic bond, with the departure of an electron pair. This group may thus be easily replaced by another group during a substitution reaction, for example. Such leaving groups are, for example, halogens or an activated hydroxyl group such as a methanesulfonate, benzenesulfonate, p-toluenesulfonate, triflate, acetate, and the like. Examples of leaving groups and references for their preparation are given in “Advances in Organic Chemistry”, J. March, 3^{rd} Edition, Wiley Interscience, 1985, p. 310-316;
a coupling catalyst: a complex of metals such as palladium and nickel which are generally used in a catalytic quantity allowing the formation of carbon-carbon bonds from halogenated derivatives and organometallic compounds derived from tin (via a “Stille coupling”), from magnesium (via a “Corriu-Kumada coupling”), from boron (via a “Suzuki coupling”), from zinc (via a “Negishi coupling”), and the like. Examples of such coupling catalysts are described in “Palladium reagents and catalysts—Innovations in organic synthesis”, J. Tsuji. (John Wiley & Sons, Inc., Chichester), 1995.

Among the compounds of formula (I) which are the subject of the invention, there may be mentioned a group of compounds in which:
n represents 0, 1, 2 or 3;
   and/or
n′ represents 0, 1, 2, 3 or 4;
   and/or
R1 represents an alkyl group, advantageously a —(C1-C6)alkyl group, more advantageously a —(C1-C4)alkyl group; and/or
R2 represents:
   a cycloalkyl group, advantageously a —(C3-C10)cycloalkyl group, more advantageously still a —(C3-C5)cycloalkyl group,
   an alkyl group, advantageously a —(C1-C6)alkyl group, more advantageously still a —(C1-C4)alkyl group, or
   an alkoxy group, advantageously a —O—(C1-C6)alkyl group, more advantageously still a —O—(C1-C4)alkyl group,
said cycloalkyl, alkyl or alkoxy groups being optionally substituted with one or more halogen atoms, advantageously with one or more fluorine atoms;
   advantageously R2 represents an alkyl group;
   and/or
R3 represents:
   a hydrogen atom, or
   a —C(O)alkyl group, advantageously a group

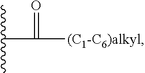

more advantageously still a group

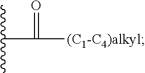

advantageously R3 represents a hydrogen atom;
and/or
Ar represents a 5- or 6-membered aryl or heteroaryl ring,
   and/or
Y, Z, V and W:
   a) represent, independently of each other,
      i) a ═CH— group,
      ii) a ═C(R5)- group in which R5 represents:
         an alkyl group, advantageously a —(C1-C6)alkyl group, more advantageously still a —(C1-C4)alkyl group,
         a halogen atom, advantageously chosen from the fluorine atom or the chlorine atom, or
         an alkoxy group, advantageously a —O—(C1-C6)alkyl group, more advantageously still a —O—(C1-C4)alkyl group,
      iii) a heteroatom chosen from the nitrogen atom, the sulfur atom and the oxygen atom, and/or
   b) at most one among Y, Z, V and W is optionally absent,
      it being understood that when Y, Z, V and W are contained in a heteroaryl chosen from pyrrolyl, imidazolyl, pyrazolyl and triazolyls, at least one of the nitrogen atoms of said heteroaryl may be optionally substituted with a group R6,
      and/or
R4 represents a group chosen from:
   the alkyl groups, advantageously a-(C1-C6)alkyl group, more advantageously still a —(C1-C4)alkyl group;
   the alkoxyalkyl groups,
   the groups —NRR′,
   the cycloalkyl groups, advantageously the —(C3-C5)cycloalkyl groups,
   the alkenyl groups, advantageously the C2-C10 groups comprising at least one C═C double bond, more advantageously still the C2-C6 groups comprising at least one C═C double bond,
   the aryl groups, advantageously the aryl groups comprising 6 carbon atoms, said aryl groups being optionally substituted with at least one halogen atom, advantageously chosen from the fluorine and chlorine atoms, and/or with at least one group chosen from the —(C1-C5)alkyl, haloalkyl, nitrile, haloalkyloxy, alkoxy and nitro groups and the groups —NRR′,
   the heteroaryl groups, advantageously the 5- or 6-membered heteroaryl groups comprising at least one heteroatom chosen from the nitrogen or sulfur atom, said heteroaryl groups being optionally substituted with at least one group chosen from the alkyl groups, advantageously a —(C1-C4)alkyl group and the heterocycloalkyl groups comprising at least one heteroatom chosen from the nitrogen and oxygen atoms; advantageously said heterocycloalkyl group is morpholinyl;
   (i) it being understood that when said heteroaryl groups are chosen from pyrrolyl, imidazolyl, pyrazolyl and triazolyls, at least one of the nitrogen atoms of said heteroaryl may be optionally substituted with a group R6,
   (ii) advantageously said heteroaryl groups are chosen from the pyridinyl and imidazolyl groups,
the heterocycloalkyl groups comprising at least one heteroatom chosen from the nitrogen, sulfur and oxygen atoms and being optionally substituted with at least one substituent chosen from (i) the halogen atoms, advantageously chosen from the fluorine and chlorine atoms, (ii) the haloalkyl groups, advantageously the fluoro(C1-C4)alkyl groups substituted with 1, 2, 3, 4 or 5 fluorine atoms, (iii) the alkyl groups, advantageously the linear or branched —(C1-C4)alkyl groups, and (iv) the cycloalkyl groups, advantageously the —(C3-C5)cycloalkyl groups, it being understood that when the heterocycloalkyl groups are chosen from pyrrolinyl, pyrrolidinyl, imidazolidinyl, imidazolinyl, pyrazolinyl, pyrazolidinyl, piperidinyl, morpholinyl, piperazinyl and thiomorpholinyl, at least one of the nitrogen atoms of said heterocycloalkyl may be optionally substituted with a group R6,
   and/or
R5 represents:
   an alkyl group, advantageously a —(C1-C6)alkyl group, more advantageously still a —(C1-C4)alkyl group,
   a halogen atom, advantageously chosen from the fluorine atom and the chlorine atom, or
   an alkoxy group, advantageously a —O—(C1-C6)alkyl group, more advantageously still a —O—(C1-C4)alkyl group, and/or
R6 represents a group chosen from an alkyl group, advantageously a —(C1-C6)alkyl group, more advantageously still a —(C1-C4)alkyl group,
   and/or
R and R′, which may be identical or different, representing, independently of each other, a hydrogen atom, an alkyl group or a —(C3-C6)cycloalkyl group,
   in the form of an acid, a base or an addition salt with an acid or a base.

Among the compounds of formula (I) which are the subject of the invention, there may be mentioned a group of compounds in which:

R1 represents a —(C1-C4)alkyl group,
and/or
R2 represents a —(C1-C4)alkyl group,
and/or
n′ represents 1,
and/or
R3 represents a hydrogen atom,
and/or
Ar represents a phenyl,
and/or
said compounds are in the form of a base or of addition salts with an acid, advantageously hydrochloric acid.

Among the compounds of formula (I) which are the subject of the invention, there may be mentioned a group of compounds in which:

R4 represents a group chosen from:
a the alkyl groups, advantageously a —(C1-C6)alkyl group, more advantageously still a —(C1-C4)alkyl group;
the groups —NRR′, with R and R′, which may be identical or different, representing, independently of each other, a hydrogen atom, an alkyl group or a —(C3-C6)cycloalkyl group,
a the alkenyl groups, advantageously the C2-C10 groups comprising at least one C═C double bond, more advantageously still the C2-C6 groups comprising at least one C═C double bond,
the aryl groups, advantageously the aryl groups comprising 6 carbon atoms, said aryl groups being optionally substituted with at least one halogen atom, advantageously chosen from the fluorine and chlorine atoms, and/or with at least one group chosen from alkoxy groups and the groups —NRR′, with R and R′ as defined above,
the heteroaryl groups, advantageously the 5- or 6-membered heteroaryl groups comprising at least one heteroatom chosen from the nitrogen atom, said heteroaryl groups being optionally substituted with at least one group chosen from alkyl groups, advantageously a —(C1-C4)alkyl group, and heterocycloalkyl groups comprising at least one heteroatom chosen from the nitrogen and oxygen atoms; advantageously said heterocycloalkyl group is morpholinyl;
   it being understood that when said heteroaryl groups are chosen from pyrrolyl, imidazolyl, pyrazolyl and triazolyls, at least one of the nitrogen atoms of said heteroaryl may be optionally substituted with a group R6, with R6 representing a group chosen from an alkyl group, advantageously a —(C1-C6)alkyl group, more advantageously still a —(C1-C4)alkyl group.

Among the compounds of formula (I) which are the subject of the invention, there may be mentioned a group of compounds in which R4 represents a group chosen from the phenyl, pyridinyl and imidazolyl groups.

Among the compounds of formula (I) which are the subject of the invention, there may be mentioned a group of compounds in which Y, Z, V and W each represents a ═CH group and/or a ═C(R5)- group, with R5 representing a chlorine or fluorine atom, Y, Z, V and W thus being in an optionally substituted phenyl group.

Among the compounds of formula (I) which are the subject of the invention, the following compounds may be mentioned in particular:
2-amino-1-ethyl-7-(3-fluoro-4-{[(pyridin-3-ylmethyl)sulfonyl]amino}phenyl)-N-methyl-4-oxo-1,4-dihydro-1,8-naphthyridine-3-carboxamide (compound No. 1)
2-amino-1-ethyl-7-(3-fluoro-4-{[(3-fluorophenyl)sulfonyl]amino}phenyl)-N-methyl-4-oxo-1,4-dihydro-1,8-naphthyridine-3-carboxamide (compound No. 2)
2-amino-7-{4-[(ethenylsulfonyl)amino]-3-fluorophenyl}-1-ethyl-N-methyl-4-oxo-1,4-dihydro-1,8-naphthyridine-3-carboxamide (compound No. 3)
2-amino-7-[4-({[2-(dimethylamino)ethyl]sulfonyl}amino)-3-fluorophenyl]-1-ethyl-N-methyl-4-oxo-1,4-dihydro-1,8-naphthyridine-3-carboxamide (compound No. 4)
2-amino-7-(4-{[(3-aminobenzyl)sulfonyl]amino}-3-fluorophenyl)-1-ethyl-N-methyl-4-oxo-1,4-dihydro-1,8-naphthyridine-3-carboxamide (compound No. 5)
2-amino-1-ethyl-7-(3-fluoro-4-{[(1-methyl-1H-imidazol-4-yl)sulfonyl]amino}phenyl)-N-methyl-4-oxo-1,4-dihydro-1,8-naphthyridine-3-carboxamide (compound No. 6)
2-amino-7-{4-[(butylsulfonyl)amino]-3-fluorophenyl}-1-ethyl-N-methyl-4-oxo-1,4-dihydro-1,8-naphthyridine-3-carboxamide (compound No. 7)
2-amino-7-(3-chloro-4-{[(2,3-dichlorophenyl)sulfonyl]amino}phenyl)-1-ethyl-N-methyl-4-oxo-1,4-dihydro-1,8-naphthyridine-3-carboxamide (compound No. 8)
2-amino-7-(4-{[(2,5-dichlorophenyl)sulfonyl]amino}-3-fluorophenyl)-1-ethyl-N-methyl-4-oxo-1,4-dihydro-1,8-naphthyridine-3-carboxamide (compound No. 9)
2-amino-1-ethyl-N-methyl-4-oxo-7-{4-[(pyridin-3-ylsulfonyl)amino]phenyl}-1,4-dihydro-1,8-naphthyridine-3-carboxamide (compound No. 10)
2-amino-7-(4-{[(2,6-dichlorophenyl)sulfonyl]amino}-3-fluorophenyl)-1-ethyl-N-methyl-4-oxo-1,4-dihydro-1,8-naphthyridine-3-carboxamide (compound No. 11)
2-amino-7-(2-chloro-4-{[(2,5-dichlorophenyl)sulfonyl]amino}phenyl)-1-ethyl-N-methyl-4-oxo-1,4-dihydro-1,8-naphthyridine-3-carboxamide (compound No. 12)
2-amino-7-(2-chloro-4-{[(2,3-dichlorophenyl)sulfonyl]amino}phenyl)-1-ethyl-N-methyl-4-oxo-1,4-dihydro-1,8-naphthyridine-3-carboxamide (compound No. 13)
2-amino-7-(4-{[(2,3-dichlorophenyl)sulfonyl]amino}-2-fluorophenyl)-1-ethyl-N-methyl-4-oxo-1,4-dihydro-1,8-naphthyridine-3-carboxamide (compound No. 14)
2-amino-7-(4-{[(2,3-dichlorophenyl)sulfonyl]amino}-3-methylphenyl)-1-ethyl-N-methyl-4-oxo-1,4-dihydro-1,8-naphthyridine-3-carboxamide (compound No. 15)
2-amino-1-ethyl-N-methyl-7-{4-[(methylsulfonyl)amino]phenyl}-4-oxo-1,4-dihydro-1,8-naphthyridine-3-carboxamide (compound No. 16)
2-amino-7-(4-{[(2,3-dichlorophenyl)sulfonyl]amino}phenyl)-1-ethyl-N-methyl-4-oxo-1,4-dihydro-1,8-naphthyridine-3-carboxamide (compound No. 17)
2-amino-7-(4-{[(2,3-dichlorophenyl)sulfonyl]amino}-3-fluorophenyl)-1-ethyl-N-methyl-4-oxo-1,4-dihydro-1,8-naphthyridine-3-carboxamide (compound No. 18)
2-amino-7-(4-{[(2-chlorophenyl)sulfonyl]amino}phenyl)-1-ethyl-N-methyl-4-oxo-1,4-dihydro-1,8-naphthyridine-3-carboxamide (compound No. 19)
2-amino-1-ethyl-7-(3-fluoro-4-{[(2-fluorophenyl)sulfonyl]amino}phenyl)-N-methyl-4-oxo-1,4-dihydro-1,8-naphthyridine-3-carboxamide (compound No. 20)
2-amino-7-(4-{[(4-chlorophenyl)sulfonyl]amino}-3-fluorophenyl)-1-ethyl-N-methyl-4-oxo-1,4-dihydro-1,8-naphthyridine-3-carboxamide (compound No. 21)
2-amino-7-(4-{[(3-chlorophenyl)sulfonyl]amino}-3-fluorophenyl)-1-ethyl-N-methyl-4-oxo-1,4-dihydro-1,8-naphthyridine-3-carboxamide (compound No. 22)
2-amino-7-(4-{[(3,4-difluorophenyl)sulfonyl]amino}-3-fluorophenyl)-1-ethyl-N-methyl-4-oxo-1,4-dihydro-1,8-naphthyridine-3-carboxamide (compound No. 23)
2-amino-1-ethyl-7-(3-fluoro-4-{[(4-fluorophenyl)sulfonyl]amino}phenyl)-N-methyl-4-oxo-1,4-dihydro-1,8-naphthyridine-3-carboxamide (compound No. 24)
2-amino-1-ethyl-7-(3-fluoro-4-{[(3-methoxyphenyl)sulfonyl]amino}phenyl)-N-methyl-4-oxo-1,4-dihydro-1,8-naphthyridine-3-carboxamide (compound No. 25)
2-amino-1-ethyl-7-[3-fluoro-4-({[6-(morpholin-4-yl)pyridin-3-yl]sulfonyl}amino)phenyl]-N-methyl-4-oxo-1,4-dihydro-1,8-naphthyridine-3-carboxamide (compound No. 26)
2-amino-1-ethyl-7-(3-fluoro-4-{[(pyridin-2-ylmethyl)sulfonyl]amino}phenyl)-N-methyl-4-oxo-1,4-dihydro-1,8-naphthyridine-3-carboxamide (compound No. 27).

It should be noted that the above compounds were named in the IUPAC nomenclature with the aid of the software ACDLABS 10.0 ACD/name (Advanced Chemistry Development).

In accordance with the invention, the compounds of the general formula (I) may be prepared according to the following method.

According to scheme 1, a 2,6-dihalonicotinic acid of formula (II), where X and X′ represent, independently of each other, a halogen atom advantageously chosen from the F, Cl and Br atoms, advantageously X and X′ represent a chlorine atom, is mono-substituted at the 2 position with an amine of formula R2-(CH2)n′-NH₂ in which R2 and n′ are as previously defined in relation to the compounds of formula (I), which are the subject of the invention. This reaction may take place at room temperature, or at a temperature of 50° C. to 100° C., with conventional or microwave heating and in a protic solvent such as an alcohol, for example ethanol, n-butanol, tert-butanol or water. The acid (III), obtained from step (i), is then activated to a derivative of formula (IV).

This derivative (IV) may either be in the form of an acid fluoride with A=F by the action of cyanuryl fluoride at room temperature, in the presence of a base such as triethylamine or pyridine and in a solvent such as dichloromethane or THF, as described by G. OLAH et al. in Synthesis (1973), 487, or in the form of an imidazolide with A=imidazolyl by the action of carbodiimidazole in a solvent such as DMF or THF or by other methods known to a person skilled in the art, such as those described by MUKAIYAMA and TANAKA in Chem. Lett. (1976), 303 or by ISHIKAWA and SASAKI in Chem. Lett. (1976), 1407.

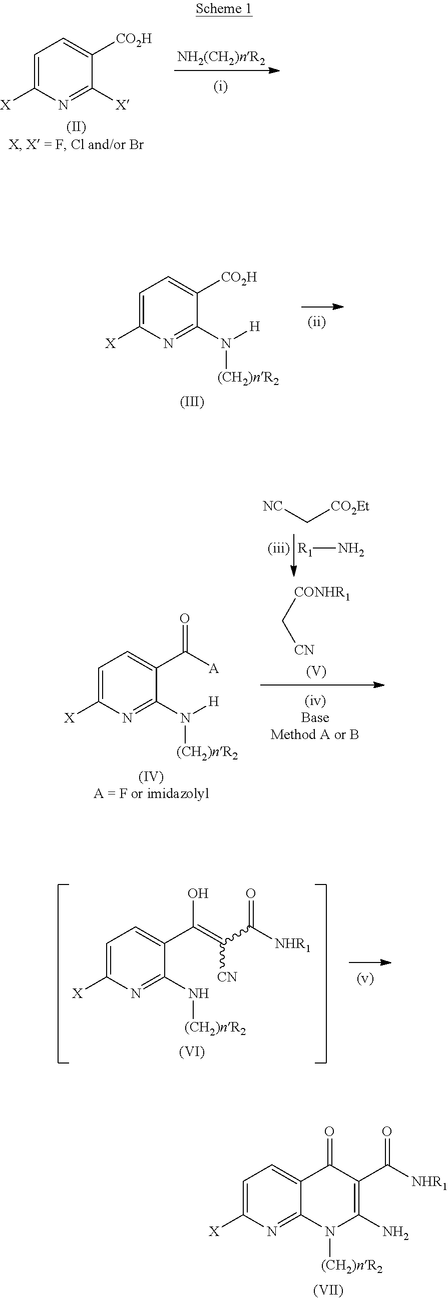

The acid fluoride (compound of formula (IV) with A=F, X=halogen, advantageously X═Cl and with n′ and R2 as defined above) or the imidazolide (compound of formula (IV) with A=imidazolyl, X=halogen, advantageously X═Cl and with n′ and R2 as defined above) of formula (IV) obtained at the end of step (ii) are very reactive but stable. They may then be reacted with an N-substituted cyanoacetamide of formula (V) according to the methods A or B described below.

According to the method A, two equivalents of a base such as sodium hydride or potassium tert-butoxide are used for step (iv) for condensation of the N-substituted cyanoacetamide derivative (V), with a compound of formula (IV); after leaving overnight at room temperature, a β-ketocyanoacetamide of formula (VI) is obtained, which is then cyclized to a pyridinopyridinone of formula (VII) in which X=halogen, advantageously X═Cl and R1, R2, n′ are as defined above by heating to a temperature between 90 and 125° C. in a polar solvent such as n-butanol, DMSO or DMF.

The method B is similar to method A for the condensation step (iv) but a third equivalent of the based used is added to the reaction mixture, and the compound of formula (VI) formed undergoes cyclization in situ, at room temperature, to directly give the pyridinopyridinone compound of formula (VII) in which X=halogen, advantageously X═Cl and R1, R2, n′ are as defined above.

The N-alkylcyanoacetamides of formula (V) are prepared according to step (iii) by reacting ethyl cyanoacetate with an excess of amine of formula R₁—NH₂ (where R₁ is as previously defined in relation to the compounds of formula (I) which are the subject of the invention) in a solvent such as THF or ethanol, at a temperature ranging from room temperature to the reflux temperature of the solvent.

To obtain the compounds of formula (I) which are the subject of the present invention, two methods can be used starting with the halogenated intermediate of formula (VII) previously described.

According to the route 1 represented in scheme 2, the intermediate (VII) in which X represents a leaving group, advantageously a halogen atom, advantageously an atom chosen from F, Cl and Br, more advantageously still a Cl atom, and in which n′, R1 and R2 are as defined above in accordance with the invention, is used in step (vi) in a SUZUKI coupling reaction with a boronic acid or a boronic ester of bispinacol (IXa) in which n, R3, R4, V, W, Y and Z are as previously defined in relation to the compounds of formula (I) which are the subject of the invention, M being as defined in scheme 2 and it being understood that the ring (Ar), defined above in accordance with the invention, should comprise 5 or 6 members. This reaction (vi) is carried out in the presence of a catalyst such as a complex of palladium (at the oxidation state (0) or (II)) such as for example Pd(PPh₃)₄, PdCl₂(PPh₃)₂, Pd₂ dba₃, Xphos or PdCl₂(dppf), in a nonprotic or protic polar solvent such as DME, ethanol, DMF, dioxane, or mixtures of these solvents, in the presence of a base such as cesium carbonate, aqueous sodium hydrogen carbonate, or K₃PO₄, with conventional heating between 80 and 120° C. or else under the action of microwave heating between 130 and 170° C.

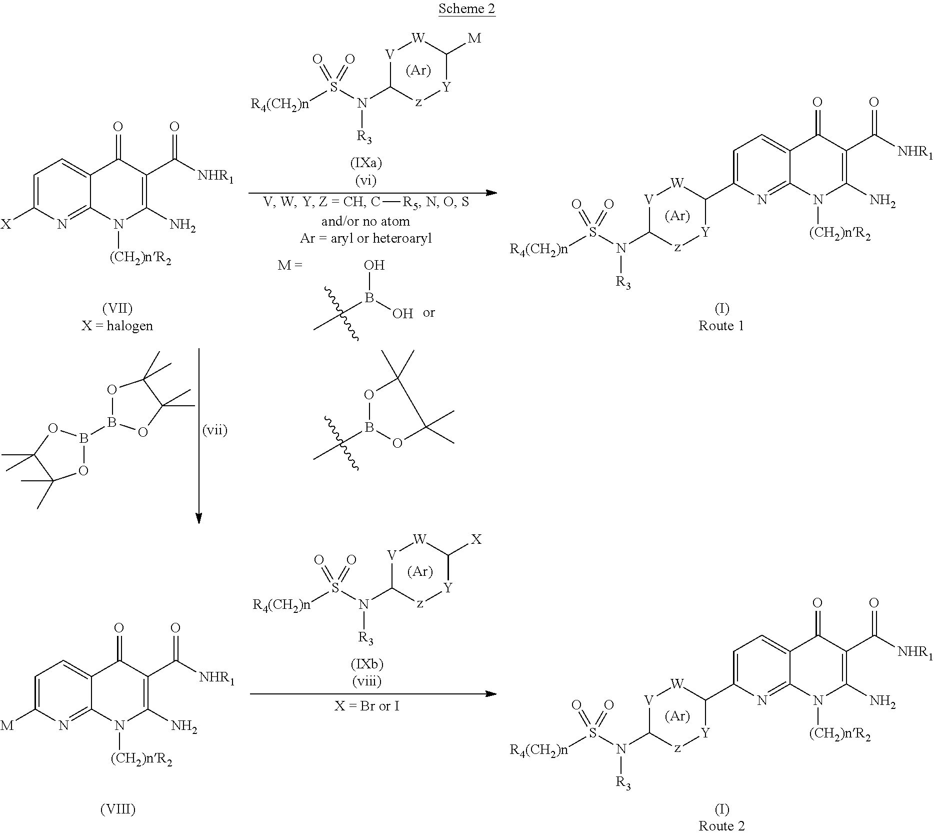

For the production of the compounds of formula (I) which are the subject of the present invention, a second route may be used starting with the halogenated intermediate of formula (VII): this route 2 is described in scheme 2. The halogenated intermediate of formula (VII), as previously defined, may be converted to a boronic acid of formula (VIII), in which M is as defined in scheme 2 and R1, R2, n′ is as defined above in accordance with the invention, according to step (vii), by reaction with bis(pinacolato)diborane in the presence of [1,1′-bis(diphenylphosphino)ferrocene]dichloropalladium (II) and potassium acetate or potassium carbonate in a polar solvent such as DMSO, DMF, DME or dioxane, at a temperature between 50 and 100° C., according to the methodology described by ISHIYAMA, T. et al. in J. Org. Chem., 1995, 60, 7508-7510 and GIROUX, A. et al. in Tet. Lett., 1997, 38, 3841-3844. In the following step (viii), said boronic acid compound (VIII) is used in a Suzuki type reaction, with a halogenated aromatic compound of formula (IXb) in which X represents a leaving group, advantageously a halogen atom, advantageously X is chosen from bromine and iodine atoms and where R₃, R₄, V, W, Y and Z are as previously defined in relation to the compounds of formula (I) which are the subject of the invention, it being understood that the aryl or heteroaryl ring (Ar) should comprise 5 or 6 members.

While the modes of preparation, starting compounds, reagents, such as the compounds of formula (IX), used in schemes 1 and 2 are not described, they are commercially available or else they may be prepared according to methods which are described in the literature or which are known to a person skilled in the art.

If necessary, some reactive functional groups present in the groups, such as for example in the groups R₁, R₂, R₃ and R₄, in particular in the groups R₅ and/or R₆, in accordance with the invention, may be protected during these reactions by means of protecting groups, as described in “Protective Groups in Organic Synthesis”, Green et al., 2^{nd} Edition (John Wiley & Sons, Inc., New York).

The subject of the invention, according to another of its aspects, is also the compounds of formulae (VII), (VIII), (IXa) and (IXb). These compounds are useful as intermediates for the synthesis of the compounds of formula (I).

The following examples illustrate the preparation of some compounds in accordance with the invention. These examples are not limiting and merely illustrate the present invention. The numbers for the compounds exemplified refer to those given in the table below, which illustrates the chemical structures and the physical properties of a few of the compounds according to the invention.

The following abbreviations and empirical formulae are used:

AcOEt ethyl acetate
DCM dichloromethane
° C. degrees Celsius
DME dimethoxyethane
DMF dimethylformamide
DMSO dimethylsulfoxide
h hour(s)
HCl hydrochloric acid
NaHCO₃ sodium hydrogen carbonate
Na₂SO₄ sodium sulfate
NaCl sodium chloride
NaOH sodium hydroxide
Na₂SO₄ sodium sulfate
min. minutes
ml milliliter
P₂O₅ diphosphorus pentoxide
THF tetrahydrofuran

The analytical conditions for the examples described below and the table below are as follows:

LC/UV/MS Coupling Conditions:

Conditions A:

Instrument (Micromass): HPLC chain: Gilson, mass spectrometer ZMD (Micromass).

LC/UV

Column: XTerra C18 3.5 μm (4.6×50 mm) (Waters), Column temp.:25° C.,

UV detection: 220 nm.

Gradient: 15 minutes

Eluents: A: H₂O+HCOOH 0.1%/B: CH₃CN+HCOOH 0.1%, Flow rate: 1 ml/min.

Gradient: 0 to 15 min from 5 to 95% B.

Conditions B:

Instrument (Micromass): HPLC chain: Waters, mass spectrometer platform II (Micromass).

LC/UV

Column: XTerra MSC18 3.5 μm (4.6×150 mm) (Waters), Column temp.: 20° C.,

UV detection: 220 nm.

Gradient: 11 minutes

Eluents: A: CH3COONH4 5 mM+CH3CN 3%/B: CH₃CN, Flow rate: 0.5 ml/min.

Gradient: 0 to 8 min from 10 to 90% B; 8 to 11 min 90% B.)

MS

Ionization mode: Electrospray positive mode ESI+, Mass range: 90-1500 amu or APCI+)

NMR

The ¹H NMR spectra were obtained using NMR spectrometers Bruker 200 or 400 MHz in CDCl3 or DMSO-d6, using the peak for CHCl3 or DMSO-d5 as reference. The chemical shifts δ are expressed in part per million (ppm). The signals observed are expressed as follows: s=singlet; d=doublet; t=triplet; m=unresolved complex or broad singlet; H=proton.

Melting Point

Melting points below 260° C. were measured with a Koffler stage apparatus and melting points greater than 260° C. were measured with a Buchi B-545 apparatus.

### EXAMPLE 1

### 2-amino-7-(4-{[(2,3-dichlorophenyl)sulfonyl]amino}phenyl)-1-ethyl-N-methyl-4-oxo-1,4-dihydro-1,8-naphthyridine-3-carboxamide

### 1.1: 6-chloro-2-(ethylamino)pyridine-3-carboxylic acid

A solution of 18.0 g (84.4 mmol) of 2,6-dichloronicotinic acid in 180 ml (3.45 mol) of a 70% ethylamine solution in water is heated at 50° C. for 10 hours. The excess amine is then evaporated under reduced pressure, and then a 10% aqueous acetic acid solution is added until precipitation of the product is obtained. The beige solid is drained, rinsed with cold water and dried in an oven. 10.5 g of the expected product are obtained. Yield=62%. Melting point: 158-160° C. MH⁺: 201.1 (tr: 7.7 min, condition A).

### 1.2: 6-chloro-2-(ethylamino)pyridine-3-carbonyl fluoride

To a suspension of 10.5 g (52.3 mmol) of the compound obtained at the end of step 1.1 in dichloromethane (250 ml), 4.2 ml (52.3 mmol) of pyridine and 8.4 ml (99.6 mmol) of cyanuric fluoride are successively added. The mixture is stirred for 3 hours at room temperature and then filtered. The solid is rinsed with dichloromethane (100 ml) and the filtrate is washed twice with ice cold water (60 ml). The organic phase is dried over Na₂SO₄ and then concentrated under reduced pressure. 10.44 g of product are obtained, in the form of an orange-colored oil. Yield=99%. The product is used without purification in the next step.

### 1.3: 2-cyano-N-methylacetamide

To 10.9 g (353.6 mmol) of a solution of methylamine in THF cooled to 0° C., 20 g (176.8 mmol) of ethyl cyanoacetate are added dropwise and then the reaction mixture is stirred at room temperature overnight. The solvents are evaporated under reduced pressure and the product is purified by recrystallization from toluene. 16.8 g of product are obtained, in the form of a beige solid. Yield=96%. Melting point=99° C.

### According to Method A of Scheme 1 Above (Steps 1.4 and 1.5 Below)

### 1.4: 3-[6-chloro-2-(ethylamino)pyridin-3-yl]-2-cyano-3-hydroxy-N-methylprop-2-enamide

To a solution, cooled to 0-5° C., of 9.80 g (100 mmol) of the compound obtained at the end of step 1.3, in 100 ml of anhydrous DMF, 3.98 g (100 mmol) of 60% sodium hydride in mineral oil are added in small quantities. At the end of the emission of hydrogen, the mixture is stirred for 10 minutes at room temperature and then cooled again to 0-5° C. A solution of 10.1 g (49.8 mmol) of the compound obtained at the end of step 1.2, in 60 ml of DMF, is added and the mixture is stirred at room temperature overnight and then 2.85 ml (49.8 mmol) of acetic acid are added. The DMF is evaporated under reduced pressure and then the residue is taken up in water and the product is extracted twice with a dichloromethane:methanol mixture in proportions of 95 to 5, and then once with an ethyl acetate:THF mixture (2:1). The combined organic phases are dried over MgSO₄, and then the solvents are evaporated under reduced pressure. 19.0 g of product are obtained which are used as they are in the next step.

### 1.5: 2-amino-7-chloro-1-ethyl-N-methyl-4-oxo-1,4-dihydro-1,8-naphthyridine-3-carboxamide

A solution of 19.0 g (49.8 mmol) of the crude product obtained at the end of step 1.4 in 600 ml of n-butanol is heated for 48 hours at 110° C. The solvent is evaporated under reduced pressure and the solid obtained is triturated in methanol. The solid is then filtered and dried in an oven. 7.9 g of the expected product are obtained in the form of a pale yellow solid. Yield=57%. Melting point: 283-286° C. MH⁺: 281.2 (tr=6.99 min, condition A)

### According to Method B of Scheme 1 Above (Step 1.6 Below Instead of the Previous Steps 1.4 and 1.5)

### 1.6: 2-amino-7-chloro-1-ethyl-N-methyl-4-oxo-1,4-dihydro-1,8-naphthyridine-3-carboxamide

To a solution cooled to 0-5° C. of 0.48 g (4.9 mmol) of the compound obtained at the end of step 1.3, in anhydrous DMF (7 ml), 0.4 g (9.95 mmol) of 60% sodium hydride in mineral oil are added in small portions. The mixture is stirred at this temperature for 10 minutes and then a solution of 1.0 g (4.93 mmol) of the compound obtained at the end of step 1.2 in anhydrous DMF (5 ml) is added. The reaction mixture is stirred overnight at room temperature and then 0.2 g (4.9 mmol) of 60% sodium hydride is added in small portions. The stirring is continued at this temperature for 30 minutes and then 0.56 ml (9.8 mmol) of acetic acid is added, followed by 60 ml of water and the solid is filtered, rinsed with water and then dried in an oven. 1.30 g of the expected product is obtained. Yield=94%. Melting point: 283-284° C. MH⁺: 281.2 (tᵣ=6.99 min, condition A)

### 1.7: [7-amino-8-ethyl-6-(methylcarbamoyl)-5-oxo-5,8-dihydro-1,8-naphthyridin-2-yl]boronic acid

A suspension of 8 g (0.03 mol) of the compound obtained at the end of step 1.5 or 1.6 (depending on whether method A or B was used), 8.0 g (0.03 mol) of bis(pinacolato)diborane and 8.5 g (0.08 mol) of potassium acetate in DMSO (130 ml), is degassed with argon for 15 minutes. 1.4 g (1.7 mmol) of [1,1′-bis(diphenylphosphino)ferrocene]dichloropalladium(II), complexed with dichloromethane (1:1), is added and the mixture is heated at 80° C. for 30 minutes, under argon, and then cooled and diluted with 1.11 of water and acidified to pH=4 by the addition of acetic acid (50 ml). The mixture is filtered and the black precipitate is washed with water (40 ml) and then with ether (60 ml). The black residue is taken up in 575 ml of an NaOH solution (1N) and the mixture is filtered on celite 545. The filtrate is acidified with 60 ml of acetic acid and the precipitate is filtered, washed with water and with ether and then dried in an oven. 6.85 g of product are obtained in the form of a white powder. Yield=83%. Melting point: 335° C. MH⁺: 291.2 (tr=5.3 min, condition A)

¹H NMR (250 MHZ, DMSO-d₆), δ (ppm): 11.69 (s, 1H); 11.12 (q, 1H, 4.67 Hz); 8.47 (s, 2H); 8.44 (d, 1H, 7.7 Hz); 7.9 (s, 1H); 7.75 (d, 1H, 7.7 Hz); 4.72 (m, 2H); 2.8 (d, 3H, 4.67 Hz); 1.22 (t, 3H, 6.9 Hz).

### 1.8: 2-amino-7-(4-{[(2,3-dichlorophenyl)sulfonyl]amino}phenyl)-1-ethyl-N-methyl-4-oxo-1,4-dihydro-1,8-naphthyridine-3-carboxamide

### 1.8.1: 2,3-dichloro-N-[4-(4,4,5,5-tetramethyl-1,3,2-dioxaborolan-2-yl)phenyl]benzenesulfonamide

To a solution of 1.1 g (5 mmol) of 4-(4,4,5,5-tetramethyl-1,3,2-dioxaborolan-2-yl)aniline in 10 ml of pyridine, 1.23 g (5 mmol) of 2,3-dichlorobenzenesulfonyl chloride is added in portions and the mixture is kept stirred for 15 h at room temperature. The solvent is evaporated and the residue is taken up in 20 ml of ethyl acetate, washed with 1N HCl and then with water and with a saturated aqueous sodium chloride solution. The organic phase is dried over Na₂SO₄ and evaporated to dryness. 2.1 g of the compound are obtained in the form of dark red crystals. Yield=100%. Melting point: 235° C.

¹H NMR (400 MHz; CDCl₃): δ (ppm): 1.2 (s; 12H, 7.0 (br s; 1H); 7.05 (d; 2H; 8 Hz); 7.2 (t; 1H; 8 Hz); 7.55 (d; 1H; 8 Hz); 7.6 (d; 2H; 8 Hz); 7.9 (d; 1h; 8 Hz).

### 1.8.2: 2-amino-7-(4-{[(2,3-dichlorophenyl)sulfonyl]amino}phenyl)-1-ethyl-N-methyl-4-oxo-1,4-dihydro-1,8-naphthyridine-3-carboxamide

280 mg (1 mmol) of chloronaphthyridine obtained in 1.6 and 450 mg (1.05 mmol) of the boronate obtained in the preceding step are dissolved in 12 ml of DME and 3 ml of ethanol. 8 ml of a saturated aqueous NaHCO₃ solution are added and then argon is bubbled through for 10 minutes. 85 mg (0.073 mmol) of tetrakis(triphenylphosphine)palladium(0) are then added and the reaction medium is heated to 90° C. under an argon atmosphere. After 3 h, the medium is filtered in the hot state, the precipitate obtained after cooling is filtered, washed with water and then with ethanol and finally with ethyl ether. The solid obtained is recrystallized from ethanol and dried in an oven. 205 mg of product are obtained in the form of a pale yellow powder. Yield=37%. Melting point=210° C.

NMR (200 MHz; DMSO-d₆): δ (ppm): 1.2 (t; 3H; 7 Hz); 2.8 (d; 3H, 4.5 Hz); 4.55 (q; 2H; 7 Hz); 7.2 (d; 2H; 8 Hz); 7.55 (t; 1H; 8 Hz); 7.7-7.9 (m; 4H); 8.1-8.2 (m; 3H); 8.4 (d; 1H; 8 Hz); 11.1 (q; 1H, 4.5 Hz); 11.65 (br s; 1H)

LCMS: MH⁺: 546 (tr: 6.58 min, condition B).

### EXAMPLE 2

### 2-amino-7-(4-{[(2,3-dichlorophenyl)sulfonyl]amino}-3-fluorophenyl)-1-ethyl-N-methyl-4-oxo-1,4-dihydro-1,8-naphthyridine-3-carboxamide

### According to Method a of Scheme 1 Above

### 2.1: 2-fluoro-4-(4,4,5,5-tetramethyl-1,3,2-dioxaborolan-2-yl)aniline

5.0 g (21.1 mmol) of 2-fluoro-4-iodoaniline and 5.89 g (23.2 mmol) of bis(pinacolato)diborane are dissolved in 130 ml of DMSO. 6.21 g (63.3 mmol) of potassium actetate are added and argon is bubbled through for 10 min. 1.21 g (1.50 mmol) of [1,1′-bis(diphenylphosphino)ferrocene]dichloropalladium(II), complexed with dichloromethane (1:1), is added and the mixture is heated at 85° C. for 4.5 hours, under argon, and then cooled and diluted with 500 ml of water. The mixture is extracted with ethyl acetate (3×200 ml), the organic phases are washed with water, dried over Na₂SO₄ and then evaporated to dryness. The crude product is purified by chromatography on silica (eluent: cyclohexane/ethyl acetate 90/10). 3.73 g of product are obtained in the form of a white powder. Yield=75%. Melting point: 112° C.

¹H NMR (400 MHz; CDCl₃): δ (ppm): 1.2 (s; 12H); 3.8 (br s; 2H); 6.55 (t; 1H; 7 Hz); 7.25-7.35 (m; 2H).

### 2.2: 2,3-dichloro-N-[2-fluoro-4-(4,4,5,5-tetramethyl-1,3,2-dioxaborolan-2-yl)-phenyl]benzenesulfonamide

Under an inert atmosphere, 1.24 g of 2,3-dichlorobenzenesulfonyl chloride is added in portions to a solution of 1.0 g (4.22 mmol) of the compound obtained at the end of step 2.1, in 40 ml of anhydrous pyridine, and then the reaction mixture is kept stirred for 18 hours. The solvent is evaporated and the residue is taken up in 20 ml of ethyl acetate, washed with 1N HCl and then with water and with a saturated aqueous sodium chloride solution. The organic phase is dried over Na₂SO₄ and evaporated to dryness and the residue is recrystallized from cyclohexane. 1.13 g of product is obtained in the form of white crystals. Yield: 60%. MH⁺: 445 (tr: 8.43 min, condition A).

### 2.3: 2-amino-7-(4-{[(2,3-dichlorophenyl)sulfonyl]amino}-3-fluorophenyl)-1-ethyl-N-methyl-4-oxo-1,4-dihydro-1,8-naphthyridine-3-carboxamide

1.13 g (2.53 mmol) of the compound obtained at the end of step 2.2 and 0.65 g (2.32 mmol) of chloronaphthyridine obtained in 1.6 are dissolved in 18 ml of dimethoxyethane and 7 ml of ethanol. 16 ml of a saturated aqueous NaHCO₃ solution are added and argon is bubbled through for 10 minutes. 0.134 g (0.12 mM) of tetrakis(triphenylphosphine)palladium(0) is added and the reaction medium is heated at 100° C. under argon for 4 h and is then filtered and the residue obtained after evaporation of the filtrate is triturated in water. The precipitate is filtered, washed with water and dried, and then purified by chromatography on silica, eluting with a gradient of methanol in dichloromethane. 740 mg of product are obtained in the form of a white powder. Yield: 57%. Melting point: 333° C.

NMR (200 MHz; DMSO-d₆): δ (ppm): 1.2 (t; 3H; 7 Hz); 2.7 (s; 3H); 4.5 (q; 2H; 7 Hz); 7.35 (t; 1H; 8 Hz); 7.5 (t; 1H; 8 Hz); 7.8-8.0 (m; 6H); 8.45 (d; 1H; 8 Hz); 10.8 (s; 1H); 11.1 (q; 1H, 4.5 Hz); 11.7 (br s; 1H).

LCMS: MH⁺: 563.9 (tr: 7.544 min, condition A)

### According to Method B of Scheme 1 Above

### 2.4: 2,3-dichloro-N-(2-fluoro-4-iodophenyl)benzenesulfonamide

To a solution of 2-fluoro-5-iodoaniline (2.8 g; 11.8 mmol) in 30 ml of anhydrous pyridine, 2.96 g (11.8 mmol) of 2,3-dichlorobenzenesulfonyl chloride are added in portions and the mixture is kept stirred for 24 hours at 20° C. The pyridine is evaporated, the residue is taken up in 50 ml of ethyl acetate and washed with water and then with a saturated aqueous NaCl solution. The organic phase is dried over Na₂SO₄ and then evaporated to dryness. The product is recrystallized from cyclohexane. 4.51 g of product are obtained in the form of a white powder. Yield: 86%; LCMS: (M-H)⁻: 444 (tr: 7.90 min, condition A).

### 2.5: 2-amino-7-(4-{[(2,3-dichlorophenyl)sulfonyl]amino}-3-fluorophenyl)-1-ethyl-N-methyl-4-oxo-1,4-dihydro-1,8-naphthyridine-3-carboxamide

To a solution of 1.0 g (2.24 mmol) of the compound obtained at the end of step 2.4 and 1.0 g (3.45 mmol) of boronic acid obtained at the end of step 1.7 in 16 ml of DMF, 4.5 ml of a saturated aqueous NaHCO₃ solution are added and argon is bubbled through for 10 min. 144 mg (0.16 mmol) of tris(dibenzylideneacetone)dipalladium(0) are then added and the mixture is heated at 85° C. for 5 hours. The reaction medium is filtered in the hot state and then the filtrate is evaporated to dryness and the residue is triturated in water (20 ml). The precipitate is filtered, washed with water and then dried in an oven under vacuum. The crude product is purified by chromatography on silica. 504 mg of product are obtained in the form of a white powder. Yield: 40%. NMR and LCMS are identical to those for the compound obtained by method A.

### EXAMPLE 3

### 2-amino-7-(4-{[(2,5-dichlorophenyl)sulfonyl]amino}-3-fluorophenyl)-1-ethyl-N-methyl-4-oxo-1,4-dihydro-1,8-naphthyridine-3-carboxamide

### 3.1: 2,5-dichloro-N-(2-fluoro-4-iodophenyl)benzenesulfonamide

Prepared according to the method described in 2.4 (method B) from 2.0 g (8.44 mmol) of 2-fluoro-4-iodoaniline and 2.11 g (8.44 mmol) of 2,5-dichlorobenzenesulfonyl chloride in 21 ml of pyridine.

3.20 g of product are obtained in the form of a white powder. Yield: 85%.

LCMS: (M-H): 444 (tr: 7.88 min, condition A).

### 3.2: 2-amino-7-(4-{[(2,5-dichlorophenyl)sulfonyl]amino}-3-fluorophenyl)-1-ethyl-N-methyl-4-oxo-1,4-dihydro-1,8-naphthyridine-3-carboxamide

Prepared according to the method described in 2.5 (method B) from 1.24 g (2.78 mmol) of the product obtained from step 3.1 and 1.24 g (4.28 mmol) of the boronic acid obtained from step 1.7.

610 mg of product are obtained in the form of a white powder. Yield: 39%. Melting point: 230° C.

¹H NMR (200 MHZ, DMSO-d₆): δ (ppm): 1.3 (t; 3H; 7 Hz); 2.8 (d; 3H, 4.5 Hz); 4.55 (q; 2H; 7 Hz); 7.40 (t; 1H; 8 Hz); 7.75 (s; 2H); 7.85-8.2 (m; 5H); 8.50 (d; 1H; 8 Hz); 10.85 (s; 1H); 11.05 (q; 1H, 4.5 Hz); 11.7 (br s; 1H).

LCMS: MH⁺: 564 (tr: 7.32 min, condition A).

### EXAMPLE 4

### 2-amino-1-ethyl-N-methyl-7-{4-[(methylsulfonyl)amino]phenyl}-4-oxo-1,4-dihydro-1,8-naphthyridine-3-carboxamide

### 4.1: {4-[(methylsulfonyl)amino]phenyl}boronic acid

A suspension of 1.0 g (5.77 mmol) of 4-aminobenzeneboronic acid hydrochloride in 10 ml of a saturated aqueous NaHCO₃ solution is cooled on ice and 2 ml (25.8 mmol) of methanesulfonyl chloride are added and the pH is adjusted to 7.2 by adding about 10 ml of a saturated aqueous NaHCO₃ solution and the mixture is kept stirred for 2 h at 5° C. 1 ml of methanesulfonyl chloride and 5 ml of a saturated aqueous NaHCO₃ solution are added. The temperature of the medium is allowed to rise to 20° C. and 3N HCl is added to pH=2 and then the whole is evaporated to dryness. 20 ml of water are added. The precipitate is filtered, washed with a minimum of water and then with ethyl ether. The product is dried in an oven under vacuum at 40° C. 0.45 g of white powder is obtained which is used without further purification.

### 4.2: 2-amino-1-ethyl-N-methyl-7-{4-[(methylsulfonyl)amino]phenyl}-4-oxo-1,4-dihydro-1,8-naphthyridine-3-carboxamide

0.33 g (1.16 mmol) of chloronaphthyridine obtained at the end of step 1.6 is dissolved in 16 ml of dimethoyethane and 8 ml of ethanol and nitrogen is bubbled through. The boronic acid obtained at the end of the preceding step as well as 8 ml of a saturated aqueous NaHCO₃ solution are added. 67 mg (0.06 mmol) of tetrakis(triphenylphosphine)palladium(0) are introduced and the medium is heated at 110° C. for 3 h. After cooling, the mixture is filtered on paper and the filtrate is concentrated to dryness. The residue is taken up in water and the precipitate obtained is filtered, washed with water and dried in an oven under vacuum over P2O5 and then purified by chromatography on silica (eluent: CH₂Cl₂/MeOH: 95/5). 450 mg of product are obtained in the form of a powder. Yield: (93%). Melting point: >300° C.

¹H NMR (200 MHZ, DMSO-d₆): δ (ppm): 1.3 (t; 3H; 7 Hz); 2.8 (d; 3H, 4.5 Hz); 3.1 (s; 3H); 4.6 (q; 2H; 7 Hz); 7.3 (d; 2H; 8 Hz); 7.9 (d; 1H; 8 Hz); 8.2 (d; 2H; 8 Hz); 8.50 (d; 1H; 8 Hz); 10.1 (s; 1H); 11.1 (q; 1H, 4.5 Hz); 11.7 (br s; 1H).

MH⁺: 416 (tr: 5.05 min, condition B).

### EXAMPLE 5

### 2-amino-1-ethyl-7-(3-fluoro-4-{[(pyridin-3-ylmethyl)sulfonyl]amino}phenyl)-N-methyl-4-oxo-1,4-dihydro-1,8-naphthyridine-3-carboxamide hydrochloride

### 5.1: N-[2-fluoro-4-(4,4,5,5-tetramethyl-1,3,2-dioxaborolan-2-yl)phenyl]-1-(pyridin-3-yl)methanesulfonamide

Under an inert atmosphere, 0.493 g (1.30 mmol) of pyridin-3-ylmethanesulfonyl chloride trifluoromethanesulfonate is introduced in portions into a solution of 0.28 g (1.18 mmol) of the compound obtained at the end of step 2.1, in 12 ml of anhydrous pyridine, and the reaction medium is kept stirred for 18 hours. 0.1 equivalent of sulfonyl chloride is added and the mixture is kept stirred for 24 hours. The pyridine is completely evaporated (two expulsions with toluene). The residue is redissolved in ethyl acetate, washed with water and then dried over Na₂SO₄ and concentrated to dryness. The solid obtained is recrystallized from cyclohexane. 330 mg of a white powder are isolated. Yield: 75%. Melting point: 206° C.

¹H NMR (400 MHz; DMSO-d₆): δ (ppm): 1.15 (s; 12H); 4.5 (s; 2H); 7.2-7.3 (m; 4H); 7.6 (d; 1H; 8 Hz); 8.35-8.45 (m; 2H); 9.8 (s; 1H).

### 5.2: 2-amino-1-ethyl-7-(3-fluoro-4-{[(pyridin-3-ylmethyl)sulfonyl]amino}phenyl)-N-methyl-4-oxo-1,4-dihydro-1,8-naphthyridine-3-carboxamide

This product was prepared according to the protocol described in paragraph 2.3 (method A) from 0.300 g (0.76 mmol) of the compound obtained in the preceding step and 0.195 g (0.69 mmol) of chloronaphthyridine obtained from step 1.6. 200 mg of product are obtained in the form of a white powder. Yield: 56%

¹H NMR (400 MHZ, DMSO-d₆): δ (ppm): 1.2 (t; 3H; 7 Hz); 2.7 (d; 3H, 4.5 Hz); 4.5 (m; 4H); 7.3 (m; 1H); 7.4 (t; 1H; 8 Hz); 7.7 (d; 1H; 8 Hz); 7.8-8.0 (m; 3H); 8.35 (m; 3H); 9.95 (s; 1H); 11.0 (q; 1H, 4.5 Hz); 11.6 (br s; 1H).

### 5.3: 2-amino-1-ethyl-7-(3-fluoro-4-{[(pyridin-3-ylmethyl) sulfonyl]amino}phenyl)-N-methyl-4-oxo-1,4-dihydro-1,8-naphthyridine-3-carboxamide hydrochloride

To a suspension of 0.200 g (0.39 mmol) of product obtained from the preceding step in 10 ml of dichloromethane, 0.2 ml of a 2N HCl solution in ethyl ether is added dropwise. The mixture is kept stirred for 10 minutes at 20° C. and the precipitate is filtered, washed with ethyl ether and dried in an oven under vacuum. 202 mg of product are obtained in the form of a white powder. Yield: 94%. Melting point: 220-223° C.

¹H NMR (200 MHZ, DMSO-d₆): δ (ppm): 1.3 (t; 3H; 7 Hz); 2.8 (s; 3H); 4.6 (m; 2H); 4.8 (s; 2H); 7.55 (t; 1H; 8 Hz); 7.65 (t; 1H; 8 Hz); 7.9-8.2 (m; 4H); 8.5 (d; 1H; 8 Hz); 8.7 (m; 2H); 10.1 (s; 1H); 11.1 (s; 1H); 11.7 (br s; 1H).

LCMS: MH⁺:511 (tr: 5, 68 min; condition A).

### EXAMPLE 6

### 2-amino-1-ethyl-7-(3-fluoro-4-{[(3-fluorophenyl)sulfonyl]amino}phenyl)-N-methyl-4-oxo-1,4-dihydro-1,8-naphthyridine-3-carboxamide

### 6.1: 3-fluoro-N-[2-fluoro-4-(4,4,5,5-tetramethyl-1,3,2-dioxaborolan-2-yl)phenyl]benzenesulfonamide

This product was prepared according to the protocol described in paragraph 2.2 (method A) from 0.5 g (2.11 mmol) of the compound obtained from step 2.1 and 0.635 g (3.16 mmol) of 3-fluorobenzenesulfonyl chloride. 0.524 g of product is obtained in the form of a white powder. Yield: 63%

¹H NMR (400 MHZ, DMSO-d₆): δ (ppm): 1.25 (s; 12H); 7.3-7.5 (m; 3H); 7.6-7.9 (m; 4H); 10.55 (s; 1H).

### 6.2: 2-amino-1-ethyl-7-(3-fluoro-4-{[(3-fluorophenyl)sulfonyl]amino}phenyl)-N-methyl-4-oxo-1,4-dihydro-1,8-naphthyridine-3-carboxamide

This product was prepared according to the protocol described in paragraph 2.3 (method A) from 0.496 g (1.25 mmol) of the compound obtained from the preceding step and 0.320 g (1.14 mmol) of chloronaphthyridine obtained from step 1.6. 0.287 g of product is obtained in the form of a beige powder. Yield: 49%. Melting point: 256° C.

¹H NMR (200 MHZ, DMSO-d₆): δ (ppm): 1.3 (t; 3H; 7 Hz); 2.8 (d; 3H, 4.5 Hz); 4.55 (q; 2H; 7 Hz); 7.4 (t; 1H; 8 Hz); 7.5-7.7 (m; 4H); 7.9-8.1 (m; 4H); 8.5 (d; 1H; 8 Hz); 10.6 (s; 1H); 11.1 (q; 1H, 4.5 Hz); 11.7 (br s; 1H).

LCMS: MH⁺:514 (tr: 7.38 min; condition B).

### EXAMPLE 7

### 2-amino-1-ethyl-7-(3-fluoro-4-{[(3-methoxyphenyl)sulfonyl]amino}phenyl)-N-methyl-4-oxo-1,4-dihydro-1,8-naphthyridine-3-carboxamide

### 7.1: N-[2-fluoro-4-(4,4,5,5-tetramethyl-1,3,2-dioxaborolan-2-yl)phenyl]-3-methoxybenzenesulfonamide

This product was prepared according to the protocol described in paragraph 2.2 (method A) from 0.5 g (2.11 mmol) of the compound obtained from step 2.1 and 0.674 g (3.16 mmol) of 3-methoxybenzenesulfonyl chloride. 0.454 g of product is obtained in the form of a white powder. Yield: 53%

¹H NMR (400 MHZ, DMSO-d₆): δ (ppm): 1.25 (s; 12H); 3.8 (s; 3H); 7.2 (d; 1H; 8 Hz); 7.25-7.4 (m; 5H); 7.6 (t; 1H; 8 Hz); 10.4 (s; 1H).

### 7.2: 2-amino-1-ethyl-7-(3-fluoro-4-{[(3-methoxyphenyl)sulfonyl]amino}phenyl)-N-methyl-4-oxo-1,4-dihydro-1,8-naphthyridine-3-carboxamide

This product was prepared according to the protocol described in paragraph 2.3 (method A) from 0.447 g (1.1 mmol) of the compound obtained from the preceding step and 0.280 g (1.0 mmol) of chloronaphthyridine obtained from step 1.6. 0.19 g of product is obtained in the form of a white powder. Yield: 36%. Melting point: >260° C.

¹H NMR (200 MHZ, DMSO-d₆): δ (ppm): 1.25 (t; 3H; 7 Hz); 2.8 (d; 3H, 4.5 Hz); 4.55 (q; 2H; 7 Hz); 7.2 (d; 1H; 8 Hz); 7.3-7.5 (m; 4H); 7.8-8.0 (m; 4H); 8.5 (d; 1H; 8 Hz); 10.4 (s; 1H); 11.1 (q; 1H, 4.5 Hz); 11.7 (br s; 1H).

LCMS: MH⁺: 526 (tr: 7.56 min; condition B).

### EXAMPLE 8

### 2-amino-1-ethyl-7-(3-fluoro-4-{[(4-fluorophenyl)sulfonyl]amino}phenyl)-N-methyl-4-oxo-1,4-dihydro-1,8-naphthyridine-3-carboxamide

### 8.1: 4-fluoro-N-[2-fluoro-4-(4,4,5,5-tetramethyl-1,3,2-dioxaborolan-2-yl)phenyl]benzenesulfonamide

This product was prepared according to the protocol described in paragraph 2.2 (method A) from 0.6 g (2.53 mmol) of the compound obtained from step 2.1 and 0.739 g (3.80 mmol) of 4-fluorobenzenesulfonyl chloride. 0.736 g of product is obtained in the form of a white powder. Yield: 53%

¹H NMR (400 MHZ, DMSO-d₆): δ (ppm): 1.1 (s; 12H); 7.0-7.25 (m; 5H); 7.65 (m; 2H); 10.25 (s; 1H).

### 8.2: 2-amino-1-ethyl-7-(3-fluoro-4-{[(4-fluorophenyl)sulfonyl]amino}phenyl)-N-methyl-4-oxo-1,4-dihydro-1,8-naphthyridine-3-carboxamide

This product was prepared according to the protocol described in paragraph 2.3 (method A) from 0.542 g (1.37 mmol) of the compound obtained from the preceding step and 0.350 g (1.25 mmol) of chloronaphthyridine obtained from step 1.6. 0.186 g of product is obtained in the form of a white powder. Yield: 29%. Melting point: >260° C.

¹H NMR (200 MHZ, DMSO-d₆): δ (ppm): 1.3 (t; 3H; 7 Hz); 2.8 (d; 3H, 4.5 Hz); 4.55 (q; 2H; 7 Hz); 7.3-7.45 (m; 3H); 7.8-8.1 (m; 6H); 8.5 (d; 1H; 8 Hz); 10.45 (s; 1H); 11.1 (q; 1H, 4.5 Hz); 11.7 (br s; 1H).

LCMS: MH+: 514 (tr: 7.47 min; condition B).

### EXAMPLE 9

### 2-amino-7-(4-{[(3-chlorophenyl)sulfonyl]amino}-3-fluorophenyl)-1-ethyl-N-methyl-4-oxo-1,4-dihydro-1,8-naphthyridine-3-carboxamide

### 9.1: 3-chloro-N-[2-fluoro-4-(4,4,5,5-tetramethyl-1,3,2-dioxaborolan-2-yl)phenyl]benzenesulfonamide

This product was prepared according to the protocol described in paragraph 2.2 (method A) from 0.5 g (2.11 mmol) of the compound obtained from step 2.1 and 0.579 g (2.74 mmol) of 3-chlorobenzenesulfonyl chloride. 0.408 g of product is obtained in the form of a white powder. Yield: 47%

¹H NMR (400 MHZ, DMSO-d₆): δ (ppm): 1.3 (s; 12H); 7.3-7.5 (m; 3H); 7.6-7.9 (m; 4H); 10.6 (s; 1H).

### 9.2: 2-amino-7-(4-{[(3-chlorophenyl)sulfonyl]amino}-3-fluorophenyl)-1-ethyl-N-methyl-4-oxo-1,4-dihydro-1,8-naphthyridine-3-carboxamide

This product was prepared according to the protocol described in paragraph 2.3 (method A) from 0.408 g (0.99 mmol) of the compound obtained from the preceding step and 0.253 g (0.90 mmol) of chloronaphthyridine obtained from step 1.6. 0.402 g of product is obtained in the form of a yellow powder. Yield: 84%. Melting point: >260° C.

¹H NMR (200 MHZ, DMSO-d₆): δ (ppm): 1.3 (t; 3H; 7 Hz); 2.8 (d; 3H, 4.5 Hz); 4.55 (q; 2H; 7 Hz); 7.2 (t; 1H; 8 Hz); 7.3-7.4 (m; 2H); 7.55-8.0 (m; 6H); 8.35 (d; 1H; 8 Hz); 10.45 (s; 1H); 11.1 (q; 1H, 4.5 Hz); 11.6 (br s; 1H).

LCMS: MH+: 530 (tr: 7.69 min; condition B).

### EXAMPLE 10

### 2-amino-1-ethyl-7-(3-fluoro-4-{[(2-fluorophenyl)sulfonyl]amino}phenyl)-N-methyl-4-oxo-1,4-dihydro-1,8-naphthyridine-3-carboxamide

### 10.1: 2-fluoro-N-[2-fluoro-4-(4,4,5,5-tetramethyl-1,3,2-dioxaborolan-2-yl) phenyl]benzenesulfonamide

This product was prepared according to the protocol described in paragraph 2.2 (method A) from 0.5 g (2.11 mmol) of the compound obtained from step 2.1 and 0.451 g (2.32 mmol) of 2-fluorobenzenesulfonyl chloride. 0.528 g of product is obtained in the form of a pinkish powder. Yield: 63%.

¹H NMR (400 MHZ, DMSO-d₆): δ (ppm): 1.2 (s; 12H); 7.25-7.5 (m; 5H); 7.7-7.8 (m; 2H); 11.65 (s; 1H).

### 10.2: 2-amino-1-ethyl-7-(3-fluoro-4-{[(2-fluorophenyl)sulfonyl]amino}phenyl)-N-methyl-4-oxo-1,4-dihydro-1,8-naphthyridine-3-carboxamide

This product was prepared according to the protocol described in paragraph 2.3 (method A) from 0.528 g (1.34 mmol) of the compound obtained from the preceding step and 0.341 g (1.21 mmol) of chloronaphthyridine obtained from step 1.6. 0.097 g of product is obtained in the form of a pale yellow powder. Yield: 16%. Melting point: >260° C.

¹H NMR (200 MHZ, DMSO-d₆): δ (ppm): 1.25 (t; 3H; 7 Hz); 2.8 (s; 3H); 4.5 (q; 2H; 7 Hz); 7.2-7.45 (m; 3H); 7.7-8.0 (m; 6H); 8.5 (d; 1H; 8 Hz); 10.65 (s; 1H); 11.1 (q; 1H; 4.5 Hz); 11.7 (br s; 1H).

LCMS: MH+: 514 (tr: 7.25 min; condition B).

### EXAMPLE 11

### 2-amino-7-(4-{[(2,6-dichlorophenyl)sulfonyl]amino}-3-fluorophenyl)-1-ethyl-N-methyl-4-oxo-1,4-dihydro-1,8-naphthyridine-3-carboxamide

### 11.1: 2,6-dichloro-N-(2-fluoro-4-iodophenyl)benzenesulfonamide

This product was prepared according to the protocol described in paragraph 2.4 (method B) from 2.8 g (11.81 mmol) of 2-fluoro-4-iodoaniline and 2.99 g (11.81 mmol) of 2,6-dichlorobenzenesulfonyl chloride. 4.43 g of product are obtained in the form of a yellow powder. Yield: 84%.

LCMS: MH+: 446 (tr: 7.55 min; condition A).

### 11.2: 2-amino-7-(4-{[(2,6-dichlorophenyl)sulfonyl]amino}-3-fluorophenyl)-1-ethyl-N-methyl-4-oxo-1,4-dihydro-1,8-naphthyridine-3-carboxamide

This product was prepared according to the protocol described in paragraph 2.5 (method B) from 2.0 g (4.48 mmol) of the compound obtained from the preceding step and 2.0 g (6.89 mmol) of the boronic acid obtained from step 1.7. 0.700 g of product is obtained in the form of a white powder. Yield: 28%. Melting point: 321° C.

¹H NMR (200 MHZ, DMSO-d₆): δ (ppm): 1.3 (t; 3H; 7 Hz); 2.8 (d; 3H, 4.5 Hz); 4.55 (q; 2H; 7 Hz); 7.35 (t; 1H; 8 Hz); 7.45-7.7 (m; 3H); 7.8-8.2 (m; 4H); 8.5 (d; 1H; 8 Hz); 10.9 (s; 1H); 11.1 (q; 1H, 4.5 Hz); 11.7 (br s; 1H).

LCMS: MH+: 564 (tr: 14.33 min; condition A).

### EXAMPLE 12

### 2-amino-7-(2-chloro-4-{[(2,3-dichlorophenyl)sulfonyl]amino}phenyl)-1-ethyl-N-methyl-4-oxo-1,4-dihydro-1,8-naphthyridine-3-carboxamide

### 12.1: 3-chloro-4-(4,4,5,5-tetramethyl-1,3,2-dioxaborolan-2-yl)aniline

This product was prepared according to the protocol described in paragraph 2.1 (method A) from 3.0 g (11.84 mmol) of 3-chloro-4-iodoaniline and 3.31 g (13.0 mmol) of bis(pinacolato)diborane. 1.51 g of product are isolated in the form of a white solid. Yield: 50%

¹H NMR (400 MHz; CDCl₃): δ (ppm): 1.4 (s; 12H); 3.95 (br s; 2H); 6.55 (d; 1H; 8 Hz); 6.7 (s; 1H); 7.6 (d; 1H; 8 Hz).

### 12.2: 2,3-dichloro-N-[3-chloro-4-(4,4,5,5-tetramethyl-1,3,2-dioxaborolan-2-yl)phenyl]benzenesulfonamide

This product was prepared according to the protocol described in paragraph 2.2 (method A) from 0.6 g (2.37 mmol) of the compound obtained from the preceding step and 0.593 g (2.37 mmol) of 2,3-dichlorobenzenesulfonyl chloride. 0.944 g of product is obtained in the form of a light beige powder. Yield: 86%.

¹H NMR (400 MHZ, DMSO-d₆): δ (ppm): 1.2 (s; 12H); 7.0 (d; 1H; 8 Hz); 7.1 (s; 1H); 7.5 (d; 1H; 8 Hz); 7.6 (t; 1H; 8 Hz); 7.95 (d; 1H; 8 Hz); 8.10 (d; 1H; 8 Hz); 11.25 (s; 1H).

### 12.3: 2-amino-7-(2-chloro-4-{[(2,3-dichlorophenyl)sulfonyl]amino}phenyl)-1-ethyl-N-methyl-4-oxo-1,4-dihydro-1,8-naphthyridine-3-carboxamide

This product was prepared according to the protocol described in paragraph 2.3 (method A) from 0.943 g (2.04 mmol) of the compound obtained from the preceding step and 0.515 g (1.84 mmol) of chloronaphthyridine obtained from step 1.6. 0.630 g of product is obtained in the form of a beige powder. Yield: 53%. Melting point: 239° C.

¹H NMR (200 MHZ, DMSO-d₆): δ (ppm): 1.2 (t; 3H; 7 Hz); 2.8 (d; 3H, 4.5 Hz); 4.5 (q; 2H; 7 Hz); 7.1-7.3 (m; 2H); 7.5-7.8 (m; 4H); 8.0 (s; 1H); 8.1 (d; 1H; 2 Hz); 8.5 (d; 1H; 8 Hz); 11.05 (q; 1H, 4.5 Hz); 11.3 (s; 1H) 11.7 (br s; 1H).

LCMS: MH+: 580 (tr: 7.74 min; condition A).

### EXAMPLE 13

### 2-amino-7-(4-{[(4-chlorophenyl)sulfonyl]amino}-3-fluorophenyl)-1-ethyl-N-methyl-4-oxo-1,4-dihydro-1,8-naphthyridine-3-carboxamide

### 13.1: 4-chloro-N-[2-fluoro-4-(4,4,5,5-tetramethyl-1,3,2-dioxaborolan-2-yl) phenyl]benzenesulfonamide

This product was prepared according to the protocol described in paragraph 2.2 (method A) from 0.5 g (2.11 mmol) of the compound obtained from step 2.1 and 0.468 g (2.15 mmol) of 4-chlorobenzenesulfonyl chloride. 0.645 g of product is obtained in the form of a pink powder. Yield: 75%. Melting point: 196° C.

¹H NMR (400 MHZ, DMSO-d₆): δ (ppm): 1.3 (s; 12H); 7.35-7.45 (m; 2H); 7.50 (d; 1H; 8 Hz); 7.7 (d; 2H; 8 Hz); 7.8 (d; 2H; 8 Hz); 10.55 (s; 1H).

### 13.2: 2-amino-7-(4-{[(4-chlorophenyl)sulfonyl]amino}-3-fluorophenyl)-1-ethyl-N-methyl-4-oxo-1,4-dihydro-1,8-naphthyridine-3-carboxamide

This product was prepared according to the protocol described in paragraph 2.3 (method A) from 0.613 g (1.49 mmol) of the compound obtained from the preceding step and 0.380 g (1.35 mmol) of chloronaphthyridine obtained from step 1.6. 0.407 g of product is obtained in the form of a yellow powder. Yield: 57%. Melting point: >260° C.

¹H NMR (200 MHZ, DMSO-d₆): δ (ppm): 1.3 (t; 3H; 7 Hz); 2.8 (d; 3H, 4.5 Hz); 4.55 (q; 2H; 7 Hz); 7.4 (t; 1H; 8 Hz); 7.7 (d; 2H; 8 Hz); 7.8 (d; 2H; 8 Hz); 7.8-8.1 (m; 4H); 8.5 (d; 1H; 8 Hz); 10.55 (s; 1H); 11.1 (q; 1H, 4.5 Hz); 11.7 (br s; 1H).

LCMS: MH+: 530 (tr: 7.91 min; condition A).

### EXAMPLE 14

### 2-amino-7-(4-{[(3,4-difluorophenyl)sulfonyl]amino}-3-fluorophenyl)-1-ethyl-N-methyl-4-oxo-1,4-dihydro-1,8-naphthyridine-3-carboxamide

### 14.1: 3,4-difluoro-N-[2-fluoro-4-(4,4,5,5-tetramethyl-1,3,2-dioxaborolan-2-yl)phenyl]benzenesulfonamide

This product was prepared according to the protocol described in paragraph 2.2 (method A) from 0.4 g (1.69 mmol) of the compound obtained from step 2.1 and 0.518 g (2.36 mmol) of 3,4-difluorobenzenesulfonyl chloride. 0.437 g of product is obtained in the form of a white powder. Yield: 63%. Melting point: 114° C.

¹H NMR (400 MHZ, DMSO-d₆): δ (ppm): 1.2 (s; 12H); 7.2 (m; 2H); 7.3 (d; 1H; 8 Hz); 7.5-7.65 (m; 2H); 7.7 (t; 1H; 8 Hz); 10.4 (s; 1H).

### 14.2: 2-amino-7-(4-{[(3,4-difluorophenyl)sulfonyl]amino}-3-fluorophenyl)-1-ethyl-N-methyl-4-oxo-1,4-dihydro-1,8-naphthyridine-3-carboxamide

This product was prepared according to the protocol described in paragraph 2.3 (method A) from 0.400 g (0.97 mmol) of the compound obtained from the preceding step and 0.259 g (0.92 mmol) of chloronaphthyridine obtained from step 1.6. 0.228 g of product is obtained in the form of a white powder. Yield: 46%. Melting point: >260° C.

¹H NMR (200 MHZ, DMSO-d₆): δ (ppm): 1.3 (t; 3H; 7 Hz); 2.8 (s; 3H); 4.55 (q; 2H; 7 Hz); 7.4 (t; 1H; 8 Hz); 7.5-7.7 (m; 2H); 7.8-8.1 (m; 5H); 8.5 (d; 1H; 8 Hz); 10.6 (s; 1H); 11.05 (q; 1H, 4.5 Hz); 11.7 (br s; 1H).

LCMS: MH+: 532 (tr: 7.55 min; condition A).

### EXAMPLE 15

### 2-amino-1-ethyl-7-[3-fluoro-4-({[6-(morpholin-4-yl)pyridin-3-yl]sulfonyl}amino)phenyl]-N-methyl-4-oxo-1,4-dihydro-1,8-naphthyridine-3-carboxamide hydrochloride

### 15.1: N-[2-fluoro-4-(4,4,5,5-tetramethyl-1,3,2-dioxaborolan-2-yl)phenyl]-6-(morpholin-4-yl)pyridine-3-sulfonamide

This product was prepared according to the protocol described in paragraph 5.1 from 0.5 g (2.11 mmol) of the compound obtained in step 2.1 and 0.743 g (2.74 mmol) of 6-(morpholin-4-yl)pyridine-3-sulfonyl chloride. 0.614 g of product is obtained in the form of a white powder. Yield: 70%. Melting point: 206° C.

¹H NMR (400 MHZ, DMSO-d₆): δ (ppm): 1.2 (s; 12H); 3.45 (s; 4H); 3.55 (s; 4H); 6.8 (m; 1H); 7.2 (m; 1H); 7.3 (m; 2H); 7.65 (m; 1H); 8.25 (m; 1H); 10.1 (s; 1H).

### 15.2: 2-amino-1-ethyl-7-[3-fluoro-4-({[6-(morpholin-4-yl)pyridin-3-yl]sulfonyl}amino)phenyl]-N-methyl-4-oxo-1,4-dihydro-1,8-naphthyridine-3-carboxamide

This product was prepared according to the protocol described in paragraph 2.3 (method A) from 0.599 g (1.29 mmol) of the compound obtained from the preceding step and 0.330 g (1.18 mmol) of chloronaphthyridine obtained from step 1.6. 0.360 g of product is obtained in the form of a yellow powder. Yield: 53%. Melting point: 260° C.

¹H NMR (200 MHZ, DMSO-d₆): δ (ppm): 1.25 (t; 3H; 7 Hz); 2.8 (d; 3H, 4.5 Hz); 3.5-3.75 (m; 8H); 4.55 (q; 2H; 7 Hz); 6.90 (d; 1H; 8 Hz); 7.5 (t; 1H; 8 Hz); 7.6 (brs; 1H); 7.8 (dd; 1H; 8 Hz and 2 Hz); 7.9-8.1 (m; 3H); 8.4 (d; 1H; 2 Hz); 8.5 (d; 1H; 8 Hz); 10.3 (s; 1H); 11.05 (q; 1H, 4.5 Hz); 11.7 (br s; 1H).

### 15.3: 2-amino-1-ethyl-7-[3-fluoro-4-({[6-(morpholin-4-yl)pyridin-3-yl]-sulfonyl}amino)phenyl]-N-methyl-4-oxo-1,4-dihydro-1,8-naphthyridine-3-carboxamide hydrochloride

The product obtained from the preceding reaction (0.2 g-0.34 mmol) is salified according to the protocol used in paragraph 5.3 using 0.17 ml of a 2N HCl solution in ether. 0.203 g of product is isolated in the form of a yellow powder. Yield: 95%; Melting point: >260° C.

¹H NMR (200 MHZ, DMSO-d₆): δ (ppm): 1.25 (t; 3H; 7 Hz); 2.8 (d; 3H, 4.5 Hz); 3.5-3.8 (m; 8H); 4.55 (q; 2H; 7 Hz); 6.90 (d; 1H; 8 Hz); 7.5 (t; 1H; 8 Hz); 7.8 (dd; 1H; 8 Hz and 2 Hz); 7.85-8.1 (m; 4H); 8.4 (d; 1H; 2 Hz); 8.5 (d; 1H; 8 Hz); 10.3 (s; 1H); 11.1 (q; 1H; 4.5 Hz); 11.7 (br s; 1H).

LCMS: MH+: 582 (tr: 7.07 min; condition B).

### EXAMPLE 16

### 2-amino-1-ethyl-7-(3-fluoro-4-{[(pyridin-2-ylmethyl)sulfonyl]amino}phenyl)-N-methyl-4-oxo-1,4-dihydro-1,8-naphthyridine-3-carboxamide hydrochloride

### 16.1: N-[2-fluoro-4-(4,4,5,5-tetramethyl-1,3,2-dioxaborolan-2-yl)phenyl]-1-(pyridin-2-yl)methanesulfonamide

This product was prepared according to the protocol described in paragraph 5.1 from 0.28 g (1.18 mmol) of the compound obtained from step 2.1 and 0.493 g (1.30 mmol) of pyridin-2-ylmethanesulfonyl chloride trifluoromethanesulfonate. 0.441 g of product is obtained in the form of a white powder. Yield: 95%. Melting point: 152° C.

¹H NMR (400 MHZ, DMSO-d₆): δ (ppm): 1.3 (s; 12H); 4.7 (s; 2H); 7.35-7.45 (m; 4H); 7.5 (d; 1H; 8 Hz); 7.8 (t; 1H; 8 Hz); 8.55 (d; 1H; 2 Hz); 9.95 (s; 1H).

### 16.2: 2-amino-1-ethyl-7-(3-fluoro-4-{[(pyridin-2-ylmethyl)sulfonyl]amino}phenyl)-N-methyl-4-oxo-1,4-dihydro-1,8-naphthyridine-3-carboxamide

This product was prepared according to the protocol described in paragraph 2.3 (method A) from 0.400 g (1.02 mmol) of the compound obtained from the preceding step and 0.260 g (0.93 mmol) of chloronaphthyridine obtained from step 1.6. 0.204 g of product is obtained in the form of a yellow powder. Yield: 43%.

¹H NMR (400 MHZ, DMSO-d₆): δ (ppm): 1.3 (t; 3H; 7 Hz); 2.8 (d; 3H, 4.5 Hz); 4.6 (q; 2H; 7 Hz); 4.7 (s; 2H); 7.35 (m; 1H); 7.5 (d; 1H; 8 Hz); 7.6 (t; 1H; 8 Hz); 7.8 (t; 1H; 8 Hz); 7.95-8.1 (m; 4H); 8.55 (m; 2H); 9.95 (s; 1H); 11.1 (q; 1H, 4.5 Hz); 11.7 (br s; 1H).

### 16.3: 2-amino-1-ethyl-7-(3-fluoro-4-{[(pyridin-2-ylmethyl)sulfonyl]amino}phenyl)-N-methyl-4-oxo-1,4-dihydro-1,8-naphthyridine-3-carboxamide hydrochloride

The product obtained from the preceding reaction (0.204 g-0.4 mmol) is salified according to the protocol used in paragraph 5.3 using 0.2 ml of a 2N HCl solution in ether. 0.202 g of product is isolated in the form of a yellow powder. Yield: 92%; Melting point: 260° C.

¹H NMR (200 MHZ, DMSO-d₆): δ (ppm): 1.3 (t; 3H; 7 Hz); 2.8 (d; 3H, 4.5 Hz); 4.6 (m; 2H); 4.75 (s; 2H); 7.4 (m; 1H); 7.55 (t; 1H; 8 Hz); 7.85 (t; 1H; 8 Hz); 7.9-8.1 (m; 5H); 8.55 (m; 2H); 10.0 (s; 1H); 11.1 (q; 1H, 4.5 Hz); 11.7 (br s; 1H).

LCMS: MH+: 511 (tr: 6.33 min; condition A).

### EXAMPLE 17

### 2-amino-7-{4-[(ethenylsulfonyl)amino]-3-fluorophenyl}-1-ethyl-N-methyl-4-oxo-1,4-dihydro-1,8-naphthyridine-3-carboxamide

### 17.1: N-[2-fluoro-4-(4,4,5,5-tetramethyl-1,3,2-dioxaborolan-2-yl)phenyl]ethenesulfonamide

This product was prepared according to the protocol described in paragraph 2.2 (method A) from 0.5 g (2.11 mmol) of the compound obtained from step 2.1 and 0.425 g (2.53 mmol) of 2-chloroethanesulfonyl chloride. 0.390 g of product is obtained in the form of a pinkish oil. Yield: 56%.

¹H NMR (400 MHZ, DMSO-d₆): δ (ppm): 1.4 (s; 12H); 6.2 (m; 2H); 7.0 (m; 1H); 7.5-7.7 (m; 3H); 10.2 (s; 1H).

### 17.2: 2-amino-7-{4-[(ethenylsulfonyl)amino]-3-fluorophenyl}-1-ethyl-N-methyl-4-oxo-1,4-dihydro-1,8-naphthyridine-3-carboxamide

This product was prepared according to the protocol described in paragraph 2.3 (method A) from 0.390 g (1.2 mmol) of the compound obtained in the preceding step and 0.280 g (1.0 mmol) of chloronaphthyridine obtained from step 1.6. 0.095 g of product is obtained in the form of a white powder. Yield: 21%. Melting point: >260° C.

¹H NMR (200 MHZ, DMSO-d₆): δ (ppm): 1.3 (t; 3H; 7 Hz); 2.8 (d; 3H, 4.5 Hz); 4.6 (q; 2H; 7 Hz); 6.05 (d; 1H; 12 Hz); 6.1 (d; 1H; 16 Hz); 6.85 (dd; 1H; 12 and 16 Hz); 7.5 (t; 1H; 8 Hz); 7.8-8.2 (m; 5H); 8.5 (d; 1H; 8 Hz); 10.1 (s; 1H); 11.0 (q; 1H, 4.5 Hz); 11.7 (br s; 1H).

LCMS: MH+: 446 (tr: 6.75 min; condition A).

### EXAMPLE 18

### 2-amino-7-[4-({[2-(dimethylamino)ethyl]sulfonyl}amino)-3-fluorophenyl]-1-ethyl-N-methyl-4-oxo-1,4-dihydro-1,8-naphthyridine-3-carboxamide hydrochloride

### 18.1: 2-(dimethylamino)-N-[2-fluoro-4-(4,4,5,5-tetramethyl-1,3,2-dioxaborolan-2-yl)phenyl]ethanesulfonamide

To a solution of 0.23 g (0.7 mmol) of the product obtained from step 17.1 in 7 ml of toluene, 0.35 ml of 2N dimethylamine solution in THF is added dropwise and the stirring is maintained for 3 hours at 20° C. The reaction medium is evaporated to dryness and the residue is taken up in 20 ml of ethyl acetate, washed with 20 ml of water and then dried over Na₂SO₄ and concentrated to dryness. 0.261 g of product is isolated in the form of a white wax. Yield: 100%.

¹H NMR (400 MHZ, DMSO-d₆): δ (ppm): 1.3 (s; 12H); 2.25 (s; 6H); 2.8 (m; 2H); 3.35 (m; 2H); 7.4 (d; 1H; 8 Hz); 7.5 (m; 2H).

LCMS: MH+: 373 (tr: 5.29 min; condition A)

### 18.2: 2-amino-7-[4-({[2-(dimethylamino)ethyl]sulfonyl}amino)-3-fluorophenyl]-1-ethyl-N-methyl-4-oxo-1,4-dihydro-1,8-naphthyridine-3-carboxamide

This product was prepared according to the protocol described in paragraph 2.3 (method A) from 0.261 g (0.70 mmol) of the compound obtained in the preceding step and 0.179 g (0.64 mmol) of chloronaphthyridine obtained from step 1.6. 0.200 g of product is obtained in the form of a yellow powder. Yield: 64%.

LCMS: MH+: 491 (tr: 5.22 min; condition A).

### 18.3: 2-amino-7-[4-({[2-(dimethylamino)ethyl]sulfonyl}amino)-3-fluorophenyl]-1-ethyl-N-methyl-4-oxo-1,4-dihydro-1,8-naphthyridine-3-carboxamide hydrochloride

The product obtained from the preceding reaction (0.200 g-0.41 mmol) is salified according to the protocol used in paragraph 5.3 using 0.2 ml of a 2N HCl solution in ether. 0.055 g of product is isolated in the form of a yellow powder. Yield: 25%; Melting point: 268-270° C.

¹H NMR (200 MHZ, DMSO-d₆): δ (ppm): 1.3 (t; 3H; 7 Hz); 2.25 (s; 6H); 2.8 (m; 5H); 3.3 (m; 2H); 4.6 (m; 2H); 7.55 (t; 1H; 8 Hz); 7.8-8.1 (m; 4H); 8.5 (d; 1H; 8 Hz); 11.05 (m; 1H); 11.7 (br s; 1H).

LCMS: MH+: 491 (tr: 4.99 min; condition A).

### EXAMPLE 19

### 2-amino-1-ethyl-7-(3-fluoro-4-{[(1-methyl-1H-imidazol-4-yl)sulfonyl]-amino}phenyl)-N-methyl-4-oxo-1,4-dihydro-1,8-naphthyridine-3-carboxamide hydrochloride

### 19.1: N-[2-fluoro-4-(4,4,5,5-tetramethyl-1,3,2-dioxaborolan-2-yl)phenyl]-1-methyl-1H-imidazole-4-sulfonamide

This product was prepared according to the protocol described in paragraph 2.2 (method A) from 0.5 g (2.11 mmol) of the compound obtained in step 2.1 and 0.481 g (2.53 mmol) of 1-methyl-1H-imidazole-4-sulfonyl chloride. 0.574 g of product is obtained in the form of a white powder. Yield: 71%. Melting point: 230° C.

¹H NMR (400 MHZ, DMSO-d₆): δ (ppm): 1.1 (s; 12H); 3.4 (s; 3H); 7.05 (s; 1H); 7.1 (s; 1H); 7.1 (s; 1H); 7.55 (m; 2H); 9.9 (s; 1H).

### 19.2: 2-amino-1-ethyl-7-(3-fluoro-4-{[(1-methyl-1H-imidazol-4-yl)sulfonyl]amino}phenyl)-N-methyl-4-oxo-1,4-dihydro-1,8-naphthyridine-3-carboxamide

This product was prepared according to the protocol described in paragraph 2.3 (method A) from 0.574 g (1.50 mmol) of the compound obtained from the preceding step and 0.384 g (1.37 mmol) of chloronaphthyridine obtained from step 1.6. 0.202 g of product is obtained in the form of a yellow powder. Yield: 30%.

LCMS: MH+: 500 (tr: 6.01 min; condition A).

### 19.3: 2-amino-1-ethyl-7-(3-fluoro-4-{[(1-methyl-1H-imidazol-4-yl)sulfonyl]amino}-phenyl)-N-methyl-4-oxo-1,4-dihydro-1,8-naphthyridine-3-carboxamide hydrochloride

The product obtained from the preceding reaction (0.200 g-0.40 mmol) is salified according to the protocol used in paragraph 5.3 using 0.2 ml of a 2N HCl solution in ether. 0.178 g of product is isolated in the form of a white powder. Yield: 82%; Melting point: >300° C.

¹H NMR (200 MHZ, DMSO-d₆): δ (ppm): 1.35 (t; 3H; 7 Hz); 2.8 (d; 3H, 4.5 Hz); 3.7 (s; 3H); 4.6 (m; 2H); 7.65 (t; 1H; 8 Hz); 7.8 (s; 1H); 7.85 (s; 1H); 7.9-8.1 (m; 4H); 8.5 (d; 1H; 8 Hz); 10.25 (s; 1H); 11.1 (q; 1H, 4.5 Hz); 11.7 (br s; 1H).

LCMS: MH+: 500 (tr: 6.06 min; condition A).

### EXAMPLE 20

### 2-amino-7-{4-[(butylsulfonyl)amino]-3-fluorophenyl}-1-ethyl-N-methyl-4-oxo-1,4-dihydro-1,8-naphthyridine-3-carboxamide

### 20.1: N-[2-fluoro-4-(4,4,5,5-tetramethyl-1,3,2-dioxaborolan-2-yl)phenyl]butane-1-sulfonamide

This product was prepared according to the protocol described in paragraph 2.2 (method A) from 0.400 g (1.69 mmol) of the compound obtained from step 2.1 and 0.31 ml (2.36 mmol) of butane-1-sulfonyl chloride. 0.652 g of product is obtained in the form of an orange-colored oil. Yield: 100%.

¹H NMR (400 MHZ, DMSO-d₆): δ (ppm): 0.8 (t; 3H; 7 Hz); 1.3 (s; 12H); 1.4 (m; 2H); 1.7 (m; 2H); 3.15 (m; 2H); 7.4 (d; 1H; 8 Hz); 7.5 (s; 2H); 9.8 (s; 1H).

### 20.2: 2-amino-7-{4-[(butylsulfonyl)amino]-3-fluorophenyl}-1-ethyl-N-methyl-4-oxo-1,4-dihydro-1,8-naphthyridine-3-carboxamide

This product was prepared according to the protocol described in paragraph 2.3 (method A) from 0.636 g (1.34 mmol) of the compound obtained from the preceding step and 0.326 g (1.16 mmol) of chloronaphthyridine obtained from step 1.6. 0.200 g of product is obtained in the form of a white powder. Yield: 36%. Melting point: 165-167° C.

¹H NMR (200 MHZ, DMSO-d₆): δ (ppm): 0.9 (t; 3H; 7 Hz); 1.2-1.4 (m; 5H); 1.75 (m; 2H); 2.8 (d; 3H, 4.5 Hz); 3.2 (m; 2H); 4.6 (m; 2H); 7.65 (t; 1H; 8 Hz); 7.8-8.2 (m; 4H); 8.5 (d; 1H; 8 Hz); 9.9 (s; 1H); 11.0 (q; 1H, 4.5 Hz); 11.7 (br s; 1H).

LCMS: MH+: 476 (tr: 7.43 min; condition A).

### EXAMPLE 21

### 2-amino-7-(4-{[(3-aminobenzyl)sulfonyl]amino}-3-fluorophenyl)-1-ethyl-N-methyl-4-oxo-1,4-dihydro-1,8-naphthyridine-3-carboxamide hydrochloride

### 21.1: N-[2-fluoro-4-(4,4,5,5-tetramethyl-1,3,2-dioxaborolan-2-yl)phenyl]-1-(3-nitrophenyl)methanesulfonamide

This product was prepared according to the protocol described in paragraph 2.2 (method A) from 0.400 g (1.69 mmol) of the compound obtained from step 2.1 and 0.502 g (2.02 mmol) of (3-nitrophenyl)methanesulfonyl chloride. 0.584 g of product is obtained in the form of a beige powder. Yield: 80%. Melting point: 178° C.

¹H NMR (400 MHZ, DMSO-d₆): δ (ppm): 1.3 (s; 12H); 4.8 (s; 2H); 7.3-7.45 (m; 3H); 7.65 (m; 1H); 7.8 (m; 1H); 8.2 (m; 2H); 10.0 (s; 1H).

### 21.2: 1-(3-aminophenyl)-N-[2-fluoro-4-(4,4,5,5-tetramethyl-1,3,2-dioxaborolan-2-yl)phenyl]methanesulfonamide

0.58 g (1.33 mmol) of the product obtained in the preceding step is dissolved in 6 ml of ethyl acetate and 1.38 g (6.65 mmol) of tin(II) chloride dihydrate is added at 20° C. The reaction medium is kept stirring for 15 hours and 0.276 g of tin(II) chloride dihydrate is added. The reaction medium is heated at 80° C. for 3 hours and left for 15 hours at 20° C. This solution was slowly poured over an aqueous NaHCO₃ solution (4.18 g in 34 ml of water) and the insoluble matter is filtered. The filtrate is extracted with 3×100 ml of ethyl acetate, washed with water and then dried over Na₂SO₄ and evaporated to dryness. 0.42 g of product is obtained in the form of a white wax. Yield: 78%.

¹H NMR (400 MHZ, DMSO-d₆): δ (ppm): 1.3 (s; 12H); 4.35 (s; 2H); 5.15 (s; 2H); 6.5 (m; 1H); 6.6 (m; 2H); 7.0 (m; 1H); 7.4 (m; 3H); 9.9 (s; 1H).

### 21.3: 2-amino-7-(4-{[(3-aminobenzyl)sulfonyl]amino}-3-fluorophenyl)-1-ethyl-N-methyl-4-oxo-1,4-dihydro-1,8-naphthyridine-3-carboxamide

This product was prepared according to the protocol described in paragraph 2.3 (method A) from 0.414 g (1.02 mmol) of the compound obtained from the preceding step and 0.260 g (0.93 mmol) of chloronaphthyridine obtained in step 1.6. 0.160 g of product is obtained in the form of a yellow powder. Yield: 33%.

LCMS: MH+: 525 (tr: 6.11 min; condition A).

### 21.4: 2-amino-7-(4-{[(3-aminobenzyl)sulfonyl]amino}-3-fluorophenyl)-1-ethyl-N-methyl-4-oxo-1,4-dihydro-1,8-naphthyridine-3-carboxamide hydrochloride

The product obtained from the preceding reaction (0.160 g-0.31 mmol) is salified according to the protocol used in paragraph 5.3 using 0.15 ml of a 2N HCl solution in ether. 0.100 g of product is isolated in the form of a beige powder: Yield: 58%; Melting point: >260° C.

¹H NMR (200 MHZ, DMSO-d₆): δ (ppm): 1.3 (t; 3H; 7 Hz); 2.8 (d; 3H, 4.5 Hz); 4.4 (s; 2H); 4.6 (m; 2H); 6.6-6.8 (m; 3H); 7.05 (t; 1H; 8 Hz); 7.5 (t; 1H; 8 Hz); 7.9-8.1 (m; 4H); 8.5 (d; 1H; 8 Hz); 9.9 (s; 1H); 11.1 (q; 1H, 4.5 Hz); 11.7 (br s; 1H).

LCMS: MH+: 525 (tr: 6.02 min; condition A).

### EXAMPLE 22

### 2-amino-7-(4-{[(2-chlorophenyl)sulfonyl]amino}phenyl)-1-ethyl-N-methyl-4-oxo-1,4-dihydro-1,8-naphthyridine-3-carboxamide

### 22.1: 2-chloro-N-[4-(4,4,5,5-tetramethyl-1,3,2-dioxaborolan-2-yl)phenyl]benzenesulfonamide

This product was prepared according to the protocol described in paragraph 1.8-A from 0.660 g (3.01 mmol) of 4-(4,4,5,5-tetramethyl-1,3,2-dioxaborolan-2-yl)aniline and 0.635 g (3.01 mmol) of 2-chlorobenzenesulfonyl chloride. 1.13 g of product is obtained in the form of a dark red powder. Yield: 96%. Melting point: 198° C.

¹H NMR (400 MHZ, DMSO-d₆): δ (ppm); 1.25 (s; 12H); 7.2 (d; 2H; 8 Hz); 7.4 (m; 1H); 7.5 (m; 2H); 7.6 (d; 1H; 8 Hz); 8.1 (d; 1H; 8 Hz).

### 22.2: 2-amino-7-(4-{[(2-chlorophenyl)sulfonyl]amino}phenyl)-1-ethyl-N-methyl-4-oxo-1,4-dihydro-1,8-naphthyridine-3-carboxamide

This product was prepared according to the protocol described in paragraph 2.3 (method A) from 0.413 g (1.05 mmol) of the compound obtained from the preceding step and 0.280 g (1.0 mmol) of chloronaphthyridine obtained from step 1.6. 0.180 g of product is obtained in the form of a white powder. Yield: 35%. Melting point: >260° C.

¹H NMR (200 MHZ, DMSO-d₆): δ (ppm): 1.85 (t; 3H; 7 Hz); 2.8 (d; 3H, 4.5 Hz); 4.55 (m; 2H); 7.2 (d; 2H; 8 Hz); 7.4-7.7 (m; 3H); 7.8 (d; 1H; 8 Hz); 7.8-8.2 (m; 3H); 7.9 (s; 1H); 8.45 (d; 1H; 8 Hz); 10.9 (s; 1H); 11.05 (q; 1H, 4.5 Hz); 11.7 (br s; 1H).

LCMS: MH+: 512 (tr: 6.12 min; condition B).

### EXAMPLE 23

### 2-amino-7-(3-chloro-4-{[(2,3-dichlorophenyl)sulfonyl]amino}phenyl)-1-ethyl-N-methyl-4-oxo-1,4-dihydro-1,8-naphthyridine-3-carboxamide

### 23.1: 2-chloro-4-(4,4,5,5-tetramethyl-1,3,2-dioxaborolan-2-yl)aniline

This product was prepared according to the protocol described in paragraph 2.1 (method A) from 5.0 g (24.22 mmol) of 4-bromo-2-chloroaniline and 6.76 g (26.64 mmol) of bis(pinacolato)diborane. 3.14 g of product are obtained in the form of a brown oil used in the next step without further purification.

### 23.2: 2,3-dichloro-N-[2-chloro-4-(4,4,5,5-tetramethyl-1,3,2-dioxaborolan-2-yl) phenyl]benzenesulfonamide

This product was prepared according to the protocol described in paragraph 2.2 (method A) from 1.5 g (5.92 mmol) of the compound obtained from the preceding step and 2.18 g (8.87 mmol) of 2,3-dichlorobenzenesulfonyl chloride. 1.57 g of product are obtained in the form of a beige powder. Yield: 57%. Melting point: 156° C.

### 23.3: (3-chloro-4-{[(2,3-dichlorophenyl)sulfonyl]amino}phenyl)boronic acid

0.3 g (6.09 mmol) of the product obtained from the preceding step is dissolved in 100 ml of anhydrous dichloromethane and this solution is cooled to −78° C. 20.0 ml of a 1M boron trichloride solution in dichloromethane are added dropwise and the reaction medium is kept at −78° C. for 3 hours and then for 15 hours at 20° C. At 0° C., 11 ml of methanol are added and the reaction medium is then evaporated to dryness. The residue is taken up in 20 ml of dichloromethane and extracted with a 0.5N sodium hydroxide solution and then the aqueous phase is acidified to pH=1 by addition of 1N HCl. The precipitate formed is filtered, washed with a little water and dried in an oven under vacuum over P₂O₅. 1.03 g of product is obtained in the form of a white powder. Yield: 42%. Melting point: 90° C.

¹H NMR (400 MHZ, DMSO-d₆): δ (ppm): 7.4 (d; 1H; 8 Hz); 7.55 (t; 1H; 8 Hz); 7.8 (d; 1H; 8 Hz); 7.95 (s; 1H); 8.0 (d; 1H; 8 Hz); 8.1 (d; 1H; 8 Hz); 8.3 (s; 2H); 10.6 (s; 1H).

### 23.4: 2-amino-7-(3-chloro-4-{[(2,3-dichlorophenyl)sulfonyl]amino}phenyl)-1-ethyl-N-methyl-4-oxo-1,4-dihydro-1,8-naphthyridine-3-carboxamide

This product was prepared according to the protocol described in paragraph 2.3 (method A) from 1.02 g (2.68 mmol) of the compound obtained from the preceding step and 0.565 g (2.24 mmol) of chloronaphthyridine obtained from step 1.6. 0.698 g of product is obtained in the form of a light yellow powder. Yield: 54%. Melting point: 311° C.

¹H NMR (400 MHZ, DMSO-d₆): δ (ppm): 1.3 (t; 3H; 7 Hz); 2.8 (d; 3H, 4.5 Hz); 4.55 (q; 2H; 7 Hz); 7.45 (d; 1H; 8 Hz); 7.55 (t; 1H; 8 Hz); 7.9 (d; 1H; 8 Hz); 7.95 (m; 2H); 8.0 (s; 1H); 8.15 (d; 1H; 8 Hz); 8.25 (s; 1H); 8.5 (d; 1H; 8 Hz); 10.65 (s; 1H); 11.05 (q; 1H, 4.5 Hz); 11.7 (br s; 1H).

LCMS: MH+: 582 (tr: 8.58 min; condition A).

### EXAMPLE 24

### 2-amino-7-(4-{[(2,3-dichlorophenyl)sulfonyl]amino}-2-fluorophenyl)-1-ethyl-N-methyl-4-oxo-1,4-dihydro-1,8-naphthyridine-3-carboxamide

### 24.1: 3-fluoro-4-(4,4,5,5-tetramethyl-1,3,2-dioxaborolan-2-yl)aniline

This product was prepared according to the protocol described in paragraph 2.1 (method A) from 5.0 g (26.3 mmol) of 4-bromo-3-fluoroaniline and 7.34 g (28.9 mmol) of bis(pinacolato)diborane. 2.45 g of product are obtained in the form of a brown oil which is used in the next step without further purification.

### 24.2: 2,3-dichloro-N-[3-fluoro-4-(4,4,5,5-tetramethyl-1,3,2-dioxaborolan-2-yl) phenyl]benzenesulfonamide

This product was prepared according to the protocol described in paragraph 2.2 (method A) from 2.5 g (15.4 mmol) of the compound obtained from the preceding step and 3.88 g (15.8 mmol) of 2,3-dichlorobenzenesulfonyl chloride. 2.67 g of product are obtained in the form of a beige powder. Yield: 57%.

¹H NMR (400 MHz; CDCl3): δ (ppm): 1.2 (s; 12H); 6.75 (d; 1H; 7 Hz); 6.8 (d; 1H; 12 Hz); 7.1 (s; 1H); 7.25 (t; 1H; 7 Hz); 7.5 (t; 1H; 7 Hz); 7.55 (d; 1H; 7 Hz); 7.9 (d; 1H; 7 Hz).

### 24.3: 2-amino-7-(4-{[(2,3-dichlorophenyl)sulfonyl]amino}-2-fluorophenyl)-1-ethyl-N-methyl-4-oxo-1,4-dihydro-1,8-naphthyridine-3-carboxamide

This product was prepared according to the protocol described in paragraph 2.3 (method A) from 2.6 g (5.82 mmol) of the compound obtained in the preceding step and 1.36 g (4.84 mmol) of chloronaphthyridine obtained from step 1.6. 1.89 g of product are obtained in the form of a light yellow powder. Yield: 69%. Melting point: 340° C.

¹H NMR (200 MHZ, DMSO-d₆): δ (ppm): 1.2 (t; 3H; 7 Hz); 2.8 (d; 3H, 4.5 Hz); 4.5 (q; 2H; 7 Hz); 7.05 (d; 1H; 12 Hz); 7.1 (d; 1H; 7 Hz); 7.6 (d; 1H; 7 Hz); 7.65 (d; 1H; 7 Hz); 7.8-8.2 (m; 4H); 8.15 (d; 1H; 7 Hz); 8.45 (d; 1H; 7 Hz); 11.0 (q; 1H, 4.5 Hz); 11.4 (s; 1H); 11.7 (br s; 1H).

LCMS: MH+: 564 (tr: 8.18 min; condition A).

### EXAMPLE 25

### 2-amino-7-(4-{[(2,3-dichlorophenyl)sulfonyl]amino}-3-methylphenyl)-1-ethyl-N-methyl-4-oxo-1,4-dihydro-1,8-naphthyridine-3-carboxamide

### 25.1: 2-methyl-4-(4,4,5,5-tetramethyl-1,3,2-dioxaborolan-2-yl)aniline

This product was prepared according to the protocol described in paragraph 2.1 (method A) from 5.0 g (26.8 mmol) of 4-bromo-2-methylaniline and 7.5 g (29.5 mmol) of bis(pinacolato)diborane. 0.992 g of product is obtained which is used in the next step without further purification.

¹H NMR (400 MHz; CDCl₃): δ (ppm); 1.4 (s; 12H); 2.25 (s; 3H); 3.9 (s; 2H); 6.7 (d; 1H; 7 Hz); 7.55 (m; 2H).

### 25.2: 2,3-dichloro-N-[2-methyl-4-(4,4,5,5-tetramethyl-1,3,2-dioxaborolan-2-yl)phenyl]benzenesulfonamide

This product was prepared according to the protocol described in paragraph 2.2 (method A) from 1.35 g (5.79 mmol) of the compound obtained from the preceding step and 2.13 g (8.67 mmol) of 2,3-dichlorobenzenesulfonyl chloride. 2.1 g of product are obtained in the form of a beige powder. Yield: 82%.

¹H NMR (400 MHz; CDCl3): δ (ppm): 1.2 (s; 12H); 3.15 (s; 3H); 6.8 (s; 1H); 7.1 (d; 1H; 7 Hz); 7.15 (t; 1H; 7 Hz); 7.35 (d; 1H; 7 Hz); 7.45 (s; 1H); 7.50 (d; 1H; 7 Hz); 7.9 (d; 1H; 7 Hz).

### 25.3: 2-amino-7-(4-{[(2,3-dichlorophenyl)sulfonyl]amino}-3-methylphenyl)-1-ethyl-N-methyl-4-oxo-1,4-dihydro-1,8-naphthyridine-3-carboxamide

This product was prepared according to the protocol described in paragraph 2.3 (method A) from 0.700 g (1.58 mmol) of the compound obtained from the preceding step and 0.370 g (1.32 mmol) of chloronaphthyridine obtained from step 1.6. 0.224 g of product is obtained in the form of a powder. Yield: 30%. Melting point: 299° C.

¹H NMR (200 MHZ, DMSO-d₆): δ (ppm): 1.25 (t; 3H; 7 Hz); 2.25 (s; 3H); 2.8 (d; 3H; 4.5 Hz); 4.55 (q; 2H; 7 Hz); 7.1 (d; 1H; 8 Hz); 7.5 (t; 1H; 8 Hz); 7.7-8.1 (m; 6H); 8.5 (d; 1H; 8 Hz); 10.2 (s; 1H); 11.05 (q; 1H, 4.5 Hz); 11.7 (br s; 1H).

LCMS: MH+: 560 (tr: 8.36 min; condition A).

### EXAMPLE 26

### 2-amino-7-(2-chloro-4-{[(2,5-dichlorophenyl)sulfonyl]amino}phenyl)-1-ethyl-N-methyl-4-oxo-1,4-dihydro-1,8-naphthyridine-3-carboxamide

### 26.1: 2,5-dichloro-N-[3-chloro-4-(4,4,5,5-tetramethyl-1,3,2-dioxaborolan-2-yl) phenyl]benzenesulfonamide

This product was prepared according to the protocol described in paragraph 2.2 (method A) from 0.500 g (1.97 mmol) of the compound obtained from step 12.1 and 0.484 g (1.97 mmol) of 2,5-dichlorobenzenesulfonyl chloride. 0.704 g of product is obtained in the form of a beige powder. Yield: 77%.

¹H NMR (400 MHz; CDCl3): δ (ppm): 1.25 (s; 12H); 7.0-7.15 (m; 2H); 7.50 (d; 1H; 8 Hz); 7.65 (d; 1H; 8 Hz); 7.80 (d; 1H; 8 Hz); 8.1 (s; 1H); 11.2 (s; 1H).

### 26.2: 2-amino-7-(2-chloro-4-{[(2,5-dichlorophenyl)sulfonyl]amino}phenyl)-1-ethyl-N-methyl-4-oxo-1,4-di hydro-1,8-naphthyridine-3-carboxamide

This product was prepared according to the protocol described in paragraph 2.3 (method A) from 0.703 g (1.52 mmol) of the compound obtained from the preceding step and 0.388 g (1.38 mmol) of chloronaphthyridine obtained from step 1.6. 0.224 g of product is obtained in the form of a white powder. Yield: 28%. Melting point: 323° C.

¹H NMR (200 MHZ, DMSO-d₆): δ (ppm): 1.2 (t; 3H; 7 Hz); 2.8 (d; 3H, 4.5 Hz); 4.5 (q; 2H; 7 Hz); 7.1-7.3 (m; 2H); 7.5-7.8 (m; 4H); 8.0 (s; 1H); 8.1 (s; 1H); 8.5 (d; 1H; 8 Hz); 11.0 (q; 1H, 4.5 Hz); 11.3 (s; 1H); 11.7 (br s; 1H).

LCMS: MH+: 580 (tr: 5.15 min; condition A).

The following table 1 illustrates compounds of formula (I) according to the invention for which R1 and R2 represent a methyl (abbreviated Me), n′ represents 1, R3 represents a hydrogen atom and Ar represents phenyl, these compounds are called below compounds of formula (I′).

In this table:
In the column “Form”, “-” means that the relevant compound is in the form of a free base, whereas “HCl” means that the relevant compound is in the form of a hydrochloride salt.
The symbol “ [symbol] ” means that the data are not available.

TABLE 1
(I′)

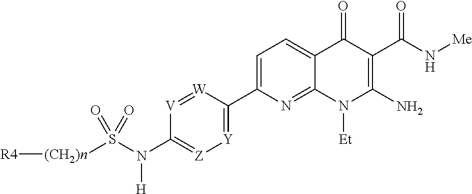

| | | | | | | | | LCMS | | Melting |
|---|---|---|---|---|---|---|---|---|---|---|
| | | | | | | | | tr min | | point |
| No. | R4 | n | V | W | Y | Z | Form | (conditions) | MH⁺ | (° C.) |
| 1 | 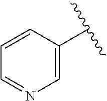 [SMILES: *c1cccnc1] | 1 | CH | CH | CH | C—F | HCl | 5.68 (A) | 511 | 220-223 |
| 2 | 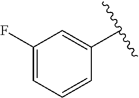 [SMILES: *c1cccc(F)c1] | 0 | CH | CH | CH | C—F | — | 7.38 (B) | 514 | 256 |
| 3 | 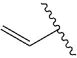 [SMILES: *C=C] | 0 | CH | CH | CH | C—F | — | 6.75 (A) | 446 | >260 |
| 4 | 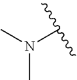 [SMILES: *N(C)C] | 2 | CH | CH | CH | C—F | HCl | 4.99 (A) | 491 | 268-270 |
| 5 | 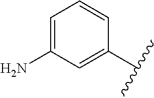 [SMILES: *c1cccc(N)c1] | 1 | CH | CH | CH | C—F | HCl | 6.02 (A) | 525 | >260 |
| 6 | 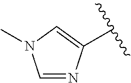 [SMILES: *c1cn(C)cn1] | 0 | CH | CH | CH | C—F | HCl | 6.06 (A) | 500 | >300 |
| 7 | Me | 3 | CH | CH | CH | C—F | — | 7.43 (A) | 476 | 165-167 |
| 8 | 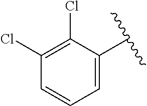 [SMILES: *c1cccc(Cl)c1Cl] | 0 | CH | CH | CH | C—Cl | — | 8.58 (A) | 582 | 311 |
| 9 | 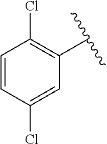 [SMILES: *c1cc(Cl)ccc1Cl] | 0 | CH | CH | CH | C—F | — | 7.32 (A) | 564 | 230 |
| 10 | 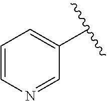 [SMILES: *c1cccnc1] | 0 | CH | CH | CH | CH | — | ¤ | ¤ | ¤ |
| 11 | 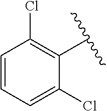 [SMILES: *c1c(Cl)cccc1Cl] | 0 | CH | CH | CH | C—F | — | 14.33 (A) | 564 | 321 |
| 12 | 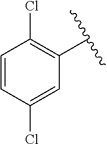 [SMILES: *c1cc(Cl)ccc1Cl] | 0 | CH | CH | C—Cl | CH | — | 5.15 (A) | 580 | 323 |
| 13 | 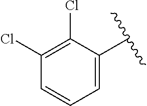 [SMILES: *c1cccc(Cl)c1Cl] | 0 | CH | CH | C—Cl | CH | — | 7.74 (A) | 580 | 239 |
| 14 | 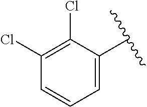 [SMILES: *c1cccc(Cl)c1Cl] | 0 | CH | CH | C—F | CH | — | 8.18 (A) | 564 | 340 |
| 15 | 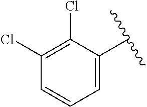 [SMILES: *c1cccc(Cl)c1Cl] | 0 | CH | CH | CH | C—CH₃ | — | 8.36 (A) | 560 | ¤ |
| 16 | Me | 0 | CH | CH | CH | CH | — | 5.05 (B) | 416 | >300 |
| 17 | 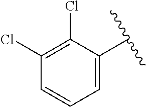 [SMILES: *c1cccc(Cl)c1Cl] | 0 | CH | CH | CH | CH | — | 6.58 (B) | 546 | 210 |
| 18 | 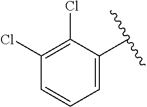 [SMILES: *c1cccc(Cl)c1Cl] | 0 | CH | CH | CH | C—F | — | 7.54 (A) | 564 | 333 |
| 19 | 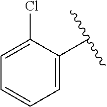 [SMILES: *c1ccccc1Cl] | 0 | CH | CH | CH | CH | — | 6.12 (B) | 512 | >260 |
| 20 | 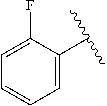 [SMILES: *c1ccccc1F] | 0 | CH | CH | CH | C—F | — | 7.25 (B) | 514 | >260 |
| 21 | 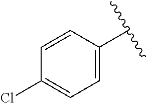 [SMILES: *c1ccc(Cl)cc1] | 0 | CH | CH | CH | C—F | — | 7.91 (A) | 530 | >260 |
| 22 | 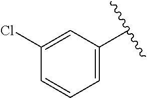 [SMILES: *c1cccc(Cl)c1] | 0 | CH | CH | CH | C—F | — | 7.69 (B) | 530 | >260 |
| 23 | 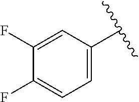 [SMILES: *c1ccc(F)c(F)c1] | 0 | CH | CH | CH | C—F | — | 7.55 (A) | 532 | >260 |
| 24 | 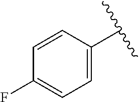 [SMILES: *c1ccc(F)cc1] | 0 | CH | CH | CH | C—F | — | 7.47 (B) | 514 | >260 |
| 25 | 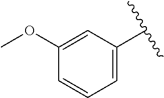 [SMILES: *c1cccc(OC)c1] | 0 | CH | CH | CH | C—F | — | 7.56 (B) | 526 | >260 |
| 26 | 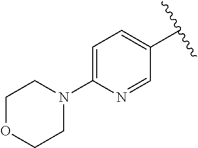 [SMILES: *c1ccc(N2CCOCC2)nc1] | 0 | CH | CH | CH | C—F | HCl | 7.07 (B) | 582 | >260 |
| 27 | 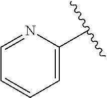 [SMILES: *c1ccccn1] | 1 | CH | CH | CH | C—F | HCl | 6.33 (A) | 511 | 260 |

The compounds according to the invention were the subject of pharmacological trials which make it possible to determine their inhibitory effect on protein kinases.

By way of example, their inhibitory effects on the p70S6 serine/threonine kinase and/or PDGF-R tyrosine kinase activity were measured in vitro in biochemical tests.

The inhibitory activity on the PDGF receptor kinases is given by the concentration which inhibits 50% of the proliferation activity of Baf3 tel/PDGF cells respectively. The inhibitory activity on p70S6 kinase is given by the concentration which inhibits 50% of the phosphorylation of the peptide substrate derived from the S6 ribosomal protein (AKRRRLSSLRA, Upstate).

Measurement of the inhibition of the PDGF beta receptor (PDGF-Rβ) tyrosine kinase activity (Baf-3 tel/PDGFRβ):

This test consists in evaluating the effects of the compounds on the PDGF beta receptor tyrosine kinase activity.

The inhibitory effect of the compounds according to the invention toward the PDGF-R receptor tyrosine kinase activity was evaluated on the hematopoietic murine cell line BaF/3 transfected with a plasmid encoding the fusion protein Tel/PDGF-R beta. This fusion protein is found in chronic myelomonocytic myeloid leukemias (CMML). It comprises the N-terminal part of the transcription factor Tel and the transmembrane and intracellular part of the PDGF-R beta receptor. This fusion protein is present in dimerized form (presence of an oligomerization domain in the N-terminal part of Tel) and therefore leads to the constitutive activity of the PDGF-R beta kinase domain. This BaF3 Tel/PDGF line has been described in the literature several times and in particular in detail in the article by CARROLL, M. et al., PNAS, 1996, 93, 14845-14850, CARROLL, M. et al., Blood 2002, 99, 14845-14850.

The BaF3 Tel/PDGF cells are washed with phosphate buffer and inoculated in 96-well plates, at the density of 5×10⁴ cells/ml (100 ml per well), in RPMI 1640 containing 10% FCS, in the presence or absence of the compounds to be tested. After 72 h of incubation, the viable cells are quantified by measuring the cellular ATP using the kit CellTiter-Glo®(Promega, Cat G7571). The cells are treated according to the instructions given by the kit supplier and the luminescence is measured with the aid of a Luminoskan (Ascent, Labsystem) with the following parameters: measurement: single; integration time: 1000 ms, lag time: 5 s.

It is thus apparent that the compounds according to the invention have an inhibitory activity on the PDGF-R beta tyrosine kinase activity. This activity is given by the concentration which inhibits 50% of the proliferation of the Baf3 tel/PDGF cells (IC₅₀). The IC₅₀ values for the compounds according to the invention are less than 10.0 μM.

For example, compounds No. 2, 18, 20 and 24 show an IC₅₀ of 36, 12, 280 and 24 nM respectively in the test for measuring the inhibitory activity of the PDGF receptor tyrosine kinase.

Measurement of the inhibition of the p70S6 kinase activity:

The active mutant recombinant S6K1 (1-421, T412E) (ref. 14-333, Upstate USA, Inc. Charlottesville Va.) (specific activity 298 U/mg) is incubated (20 mU/10 μl) with 8 concentrations of inhibitors solubilized at 1 mM in DMSO in the presence of the peptide substrate obtained from the S6 ribosomal protein (AKRRRLSSLRA, Upstate) (50 μM final) and of a cold ATP mixture (100 μM) and 1 μCi/well of [γ-33]ATP (NEN, Courtaboeuf, France). The enzyme reaction is carried out in a final volume of 50 μl in a 96-well filter plate (MultiScreen TM-PH opaque plate with Phospho-Cellulose cat # MAPHNOB, Millipore) previously soaked with 100 μl 1M Tris buffer pH 7.4 by adding the reagents of the S6 Kinase Assay kit (#17-136, Upstate) in the following order:

10 μl of 5% DMSO or various inhibitors at a 5× concentration

30 μl of reaction mixture containing the ADBI buffer (#20-108 Upstate, composed of 20 mM MOPS pH 7.2, 25 mM beta-glycerol phosphate, 5 mM EGTA, 1 mM sodium orthovanadate, 1 mM dithiothreitol), S6K1 (20 mU) and 250 μM peptide substrate [AKRRRLSSLRA] in ADBI buffer (#20-122, Upstate). The reaction is started by adding 10 μl of cold ATP/³³γATP mixture (1 μCi/50 μl as 500 μM ATP in ADBI buffer, 75 mM MgCl₂) and then incubated for 20 minutes at 30° C. before being stopped by adding 20 μl of 7.5% phosphoric acid. The reaction mixture is filtered by aspiration under vacuum (Vacuum manifold, Millipore), the wells are rinsed twice with 200 μl of 7.5% phosphoric acid (2 minutes) and then twice with 200 μl of distilled H₂O (2 minutes). After drying the plate, 25 μl/well of scintillant (Optiphase Super Mix, Wallac) are added and the radioactivity is detected with the Micro-Beta scintillation reader (Wallac). A negative control (all the reagents without peptide substrate) is prepared in order to determine the nonspecific binding of ³³γATP to the phosphocellulose filter which is subtracted from the experimental results.

It is thus apparent that the compounds of the invention have an inhibitory activity on the p70S6 kinase activity. This activity is given by the concentration which inhibits 50% of the phosphorylation of the peptide substrate derived from the S6 ribosomal protein (AKRRRLSSLRA, Upstate). The IC₅₀ values for the compounds according to the invention are less than 10.0 μM.

For example, compounds No. 8, 9, 14 and 18 showed an IC₅₀ of 412, 240, 224 and 132 nM respectively in the test for measuring the inhibitory activity of the p70S6 kinase.

The compounds according to the invention are therefore inhibitors of protein kinases, in particular PDGF tyrosine kinases receptor and, for some of them, also of p70S6 kinase.

The compounds according to the invention may therefore be used for the preparation of medicaments intended for the treatment and/or prevention of diseases linked to the activity of protein kinases, in particular of medicaments inhibiting protein kinases.

These are protein kinase-inhibiting medicaments, in particular medicaments inhibiting PDGF-R receptor tyrosine kinase and optionally p70S6 kinase.

Thus, according to another of its aspects, the subject of the invention is medicaments which comprise a compound of formula (I), or an addition salt of the latter with a pharmaceutically acceptable acid, or else a solvate of the compound of formula (I).

These medicaments find their use in therapy, in particular in the treatment and/or prevention of diseases linked to the activity of protein kinases and in particular proliferative diseases such as cancers, for example cancers of the lung (NSCLC), of the bones, of the pancreas, of the skin, Kaposi's syndrome, intraocular melanomas, cancers of the breast, of the uterus, of the cervix, of the ovaries, of the endometrium, of the vagina, of the vulva, of the urethra, of the penis, of the prostate, fallopian tube carcinomas, cancers such as GISTs and of the anal region, of the rectum, of the small intestine, of the colon, of the stomach, of the esophagus, of the endocrine, thyroid, parathyroid or adrenal glands, soft tissue sarcomas, Ewing's sarcomas, ostesarcomas, dermatofibrosarcoma and other fibrosarcomas, cancers of the bladder or of the kidney, neoplasms of the central nervous system, vertebral column and desmoid tumors, brain stem gliomas and glioblastomas, pituitary adenomas and metastases thereof, chronic or acute leukemias, lymphocytic lymphomas, Hodgkin's disease and myeloproliferative syndromes, and myelodysplastic syndromes.

Another aspect of the invention comprises a combination of at least one compound according to the invention with at least one chemotherapeutic agent.

Indeed, the compounds of the present invention may be used alone or as a mixture with at least one chemotherapeutic agent which may be chosen from cytotoxic agents and/or antiangiogenic agents. For example, the antiangiogenic agents may be a compound inhibiting VEGF-R kinase activity or a compound that is an antagonist of a growth factor.

It is also possible to combine the compounds according to the invention with a radiation treatment.

The combinations of the compounds of the invention with the chemotherapeutic agents mentioned above and/or radiation are another subject of the present invention.

The chemotherapeutic agents mentioned above and/or the radiations may be administered simultaneously, separately or sequentially. The treatment will be adapted by the practitioner according to the patient to be treated.

These medicaments also find use in therapy, in nonmalignant proliferative diseases such as for example restenosis, atherosclerosis, thrombosis, heart failure, cardiac hypertrophy, pulmonary arterial hypertension, fibrosis, diabetic nephropathy, glomerulonephritis, chronic pyelonephritis, hemangiomas, autoimmune diseases such as psoriasis, sclerodermatitis, immunosuppression (graft rejection for example).

According to another of its aspects, the present invention relates to pharmaceutical compositions comprising, as active ingredient, a compound according to the invention.

These pharmaceutical compositions contain an effective dose of at least one compound according to the invention, or a pharmaceutically acceptable salt of the latter, or else a solvate of said compound, and at least one pharmaceutically acceptable excipient.

Said excipients are chosen according to the pharmaceutical dosage form and the desired mode of administration, from the customary excipients which are known to a person skilled in the art.

In the pharmaceutical compositions of the present invention for oral, sublingual, subcutaneous, intramuscular, intravenous, topical, intratracheal, intranasal, transdermal or rectal administration, the active ingredient of formula (I) above, or its optional salt or solvate, may be administered in unit form for administration, as a mixture with conventional pharmaceutical excipients, to animals and to humans for the prophylaxis or treatment and/or the prevention of the above disorders or diseases.

The appropriate unit forms for administration comprise the forms for oral administration, such as tablets, soft or hard gelatin capsules, powders, granules and oral solutions or suspensions, forms for sublingual, buccal, intratracheal, intraocular or intranasal administration or for administration by inhalation, the forms for topical, transdermal, subcutaneous, intramuscular or intravenous administration, the forms for rectal administration and implants. For topical application, the compounds according to the invention may be used in creams, gels, ointments or lotions.

By way of example, a unit form for administration of a compound according to the invention in tablet form may comprise the following components:

| | | |
|---|---|---|
| Compound according to the invention | 50.0 | mg |
| Mannitol | 223.75 | mg |
| Croscarmellose sodium | 6.0 | mg |
| Corn starch | 15.0 | mg |
| Hydroxypropylmethylcellulose | 2.25 | mg |
| Magnesium stearate | 3.0 | mg |

The present invention, according to another of its aspects, also relates to a method for the treatment and/or prevention of the pathologies indicated above which comprises the administration, to a patient, of an effective dose of a compound according to the invention or one of its pharmaceutically acceptable salts or solvates.

The present invention, according to another of its aspects, also relates to the use of a compound of formula (I) for the preparation of a medicament intended for the treatment and/or prevention of diseases linked to the activity of protein kinases, for the treatment and/or prevention of proliferative diseases such as cancers, chronic or acute leukemias, lymphocytic lymphomas, Hodgkin's disease, and myeloproliferative syndromes, and myelodysplastic syndromes, for the treatment and/or prevention of proliferative diseases such as solid tumor cancers, for example cancers of the lung (NSCLC), of the bones, of the pancreas, of the skin, Kaposi's syndrome, intraocular melanomas, cancers of the breast, of the uterus, of the cervix, of the ovaries, of the endometrium, of the vagina, of the vulva, of the urethra, of the penis, of the prostate, fallopian tube carcinomas, cancers such as GISTs and of the anal region, of the rectum, of the small intestine, of the colon, of the stomach, of the esophagus, of the endocrine, thyroid, parathyroid or adrenal glands, soft tissue sarcomas, Ewing's sarcomas, ostesarcomas, dermatofibrosarcoma and other fibrosarcomas, cancers of the bladder or of the kidney, neoplasms of the central nervous system, vertebral column and desmoid tumors, brain stem gliomas and glioblastomas, pituitary adenomas and metastases thereof, chronic or acute leukemias, lymphocytic lymphomas, Hodgkin's disease and myeloproliferative syndromes, and myelodysplastic syndromes, or for the treatment and/or prevention of nonmalignant proliferative diseases such as restenosis, atherosclerosis, thrombosis, heart failure, cardiac hypertrophy, pulmonary arterial hypertension, fibrosis, diabetic nephropathy, glomerulonephritis, chronic pyelonephritis, hemangiomas, autoimmune diseases such as psoriasis, sclerodermatitis, immunosuppression.

We claim:

1. A compound of formula (I): 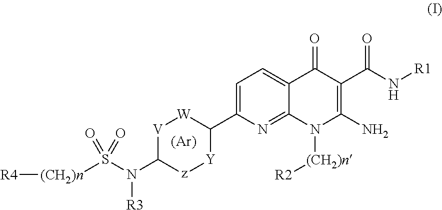
wherein:
n represents 0, 1, 2 or 3;
n′ represents 0, 1, 2, 3 or 4;
R1 represents an alkyl group;
R2 represents:
(i) a cycloalkyl group,
(ii) an alkyl group, or
(iii) an alkoxy group,
said cycloalkyl, alkyl or alkoxy groups being optionally substituted with one or more halogen atoms;
R3 represents:
i) a hydrogen atom, or
ii) a —C(O)alkyl group;
Ar represents a 5- or 6-membered aryl or heteroaryl ring wherein Y, Z, V and W:
(a) represent, independently of each other,
(i) a ═CH— group,
(ii) a ═C(R5)-group in which R5 represents:
an alkyl group,
a halogen atom, or
an alkoxy group,
(iii) a heteroatom chosen from the nitrogen atom, the sulfur atom and the oxygen atom,
(b) at most one among Y, Z, V and W being optionally absent,
it being understood that, when Ar represents a heteroaryl chosen from pyrrolyl, imidazolyl, pyrazolyl and triazolyls, at least one of the nitrogen atoms of said heteroaryl may be optionally substituted with a group R6 chosen from an alkyl group,
R4 represents a group chosen from:
an alkyl group,
an alkoxyalkyl group,
a group —NRR′ with R and R′, which may be identical or different, representing, independently of each other, a hydrogen atom, an alkyl group or a —(C3-C6)cycloalkyl group,
a cycloalkyl group,
an alkenyl group,
an aryl group, said group being optionally substituted with at least one halogen atom, or with at least one group chosen from a —(C1-C5)alkyl, haloalkyl, nitrile, haloalkyloxy, alkoxy, nitro group and the groups —NRR′ with R and R′, which may be identical or different, representing, independently of each other, a hydrogen atom or a group chosen from alkyl groups and —(C3-C6)cycloalkyl groups,
a heteroaryl group, said group comprising at least one heteroatom chosen from the nitrogen or sulfur atom, said heteroaryl groups being optionally substituted with at least one group chosen from alkyl groups and heterocycloalkyl groups comprising at least one heteroatom chosen from the nitrogen and oxygen atoms;
it being understood that, when the heteroaryl group is chosen from pyrrolyl, imidazolyl, pyrazolyl and triazolyls, at least one of the nitrogen atoms of said heteroaryl may be optionally substituted with a group R6 chosen from an alkyl group,
a heterocycloalkyl group comprising at least one heteroatom chosen from the nitrogen, sulfur and oxygen atoms and being optionally substituted with at least one substituent chosen from (i) halogen atoms, (ii) haloalkyl groups, (iii) linear or branched alkyl groups, and (iv) cycloalkyl groups,
it being understood that when the heterocycloalkyl groups are chosen from pyrrolinyl, pyrrolidinyl, imidazolidinyl, imidazolinyl, pyrazolinyl, pyrazolidinyl, piperidinyl, morpholinyl, piperazinyl and thiomorpholinyl, at least one of the nitrogen atoms of said heterocycloalkyl may be optionally substituted with a group R6 chosen from an alkyl group,
in the form of an acid, a base or an addition salt with an acid or a base.

2. The compound according to claim 1, wherein:
R1 represents a —(C1-C4)alkyl group,
in the form of a base or addition salts with an acid.

3. The compound according to claim 1, wherein:
R2 represents a —(C1-C4)alkyl group,
in the form of a base or addition salts with an acid.

4. The compound according to claim 1, wherein:
n′ represents 1,
in the form of a base or addition salts with an acid.

5. The compound according to claim 1, wherein:
R3 represents a hydrogen atom,
in the form of a base or addition salts with an acid.

6. The compound according to claim 1, wherein:
Ar represents a phenyl,
in the form of a base or addition salts with an acid.

7. The compound according to claim 1, wherein:
R4 represents a group chosen from:
an alkyl group;
a group —NRR′, with R and R′, which may be identical or different, representing, independently of each other, a hydrogen atom, an alkyl group or a —(C3-C6)cycloalkyl group,
an alkenyl group,
an aryl group, said group being optionally substituted with at least one halogen atom, and/or with at least one group chosen from alkoxy groups and the groups —NRR′, with R and R′ as defined above,
a heteroaryl group, said heteroaryl group being optionally substituted with at least one group chosen from alkyl groups and heterocycloalkyl groups comprising at least one heteroatom chosen from the nitrogen and oxygen atoms;
it being understood that, when said heteroaryl group is chosen from pyrrolyl, imidazolyl, pyrazolyl and triazolyls, at least one of the nitrogen atoms of said heteroaryl may be optionally substituted with a group R6, with R6 representing a group chosen from an alkyl group.

8. The compound according to claim 1, wherein R4 represents a group chosen from phenyl, pyridinyl and imidazolyl groups.

9. The compound according to claim 1, wherein Y, Z, V and W each represents a ═CH group or a ═C(R5)-group, and R5 represents a chlorine or fluorine atom, Y, Z, V and W being thus in an optionally substituted phenyl group.

10. The compound according to claim 1, which is:
2-amino-1-ethyl-7-(3-fluoro-4-{[(pyridin-3-ylmethyl)sulfonyl]amino}phenyl)-N-methyl-4-oxo-1,4-dihydro-1,8-naphthyridine-3-carboxamide;
2-amino-1-ethyl-7-(3-fluoro-4-{[(3-fluorophenyl)sulfonyl]amino}phenyl)-N-methyl-4-oxo-1,4-dihydro-1,8-naphthyridine-3-carboxamide;
2-amino-7-{4-[(ethenylsulfonyl)amino]-3-fluorophenyl}-1-ethyl-N-methyl-4-oxo-1,4-dihydro-1,8-naphthyridine-3-carboxamide;
2-amino-7-[4-({[2-(dimethylamino)ethyl]sulfonyl]amino)-3-fluorophenyl}-1-ethyl-N-methyl-4-oxo-1,4-dihydro-1,8-naphthyridine-3-carboxamide;
2-amino-7-(4-{[(3-aminobenzyl)sulfonyl]amino}-3-fluorophenyl)-1-ethyl-N-methyl-4-oxo-1,4-dihydro-1,8-naphthyridine-3-carboxamide;
2-amino-1-ethyl-7-(3-fluoro-4-{[(1-methyl-1H-imidazol-4-yl)sulfonyl]amino}phenyl)-N-methyl-4-oxo-1,4-dihydro-1,8-naphthyridine-3-carboxamide;
2-amino-7-{4-[(butylsulfonyl)amino]-3-fluorophenyl}-1-ethyl-N-methyl-4-oxo-1,4-dihydro-1,8-naphthyridine-3-carboxamide;
2-amino-7-(3-chloro-4-{[(2,3-dichlorophenyl)sulfonyl]amino}phenyl)-1-ethyl-N-methyl-4-oxo-1,4-dihydro-1,8-naphthyridine-3-carboxamide;
2-amino-7-(4-{[(2,5-dichlorophenyl)sulfonyl]amino}-3-fluorophenyl)-1-ethyl-N-methyl-4-oxo-1,4-dihydro-1,8-naphthyridine-3-carboxamide;
2-amino-1-ethyl-N-methyl-4-oxo-7-{4-[(pyridin-3-ylsulfonyl)amino]phenyl}-1,4-dihydro-1,8-naphthyridine-3-carboxamide;
2-amino-7-(4-{[(2,6-dichlorophenyl)sulfonyl]amino}-3-fluorophenyl)-1-ethyl-N-methyl-4-oxo-1,4-dihydro-1,8-naphthyridine-3-carboxamide;
2-amino-7-(2-chloro-4-{[(2,5-dichlorophenyl)sulfonyl]amino}phenyl)-1-ethyl-N-methyl-4-oxo-1,4-dihydro-1,8-naphthyridine-3-carboxamide;
2-amino-7-(2-chloro-4-{[(2,3-dichlorophenyl)sulfonyl]amino}phenyl)-1-ethyl-N-methyl-4-oxo-1,4-dihydro-1,8-naphthyridine-3-carboxamide;
2-amino-7-(4-{[(2,3-dichlorophenyl)sulfonyl]amino}-2-fluorophenyl)-1-ethyl-N-methyl-4-oxo-1,4-dihydro-1,8-naphthyridine-3-carboxamide;
2-amino-7-(4-{[(2,3-dichlorophenyl)sulfonyl]amino}-3-methylphenyl)-1-ethyl-N-methyl-4-oxo-1,4-dihydro-1,8-naphthyridine-3-carboxamide;
2-amino-1-ethyl-N-methyl-7-{4-[(methylsulfonyl)amino]phenyl}-4-oxo-1,4-dihydro-1,8-naphthyridine-3-carboxamide;
2-amino-7-(4-{[(2,3-dichlorophenyl)sulfonyl]amino}phenyl)-1-ethyl-N-methyl-4-oxo-1,4-dihydro-1,8-naphthyridine-3-carboxamide;
2-amino-7-(4-{[(2,3-dichlorophenyl)sulfonyl]amino}-3-fluorophenyl)-1-ethyl-N-methyl-4-oxo-1,4-dihydro-1,8-naphthyridine-3-carboxamide;
2-amino-7-(4-{[(2-chlorophenyl)sulfonyl]amino}phenyl)-1-ethyl-N-methyl-4-oxo-1,4-dihydro-1,8-naphthyridine-3-carboxamide;
2-amino-1-ethyl-7-(3-fluoro-4-{[(2-fluorophenyl)sulfonyl]amino}phenyl)-N-methyl-4-oxo-1,4-dihydro-1,8-naphthyridine-3-carboxamide;
2-amino-7-(4-{[(4-chlorophenyl)sulfonyl]amino}-3-fluorophenyl)-1-ethyl-N-methyl-4-oxo-1,4-dihydro-1,8-naphthyridine-3-carboxamide;
2-amino-7-(4-{[(3-chlorophenyl)sulfonyl]amino}-3-fluorophenyl)-1-ethyl-N-methyl-4-oxo-1,4-dihydro-1,8-naphthyridine-3-carboxamide;
2-amino-7-(4-{[(3,4-difluorophenyl)sulfonyl]amino}-3-fluorophenyl)-1-ethyl-N-methyl-4-oxo-1,4-dihydro-1,8-naphthyridine-3-carboxamide;
2-amino-1-ethyl-7-(3-fluoro-4-{[(4-fluorophenyl)sulfonyl]amino}phenyl)-N-methyl-4-oxo-1,4-dihydro-1,8-naphthyridine-3-carboxamide;
2-amino-1-ethyl-7-(3-fluoro-4-{[(3-methoxyphenyl)sulfonyl]amino}phenyl)-N-methyl-4-oxo-1,4-dihydro-1,8-naphthyridine-3-carboxamide;
2-amino-1-ethyl-7-[3-fluoro-4-({[6-(morpholin-4-yl)pyridin-3-yl]sulfonyl}amino)phenyl]-N-methyl-4-oxo-1,4-dihydro-1,8-naphthyridine-3-carboxamide; or
2-amino-1-ethyl-7-(3-fluoro-4-{[(pyridin-2-ylmethyl)sulfonyl]amino}phenyl)-N-methyl-4-oxo-1,4-dihydro-1,8-naphthyridine-3-carboxamide.

11. A process for preparing the compound according to claim 1, comprising reacting a compound of formula (IXa): 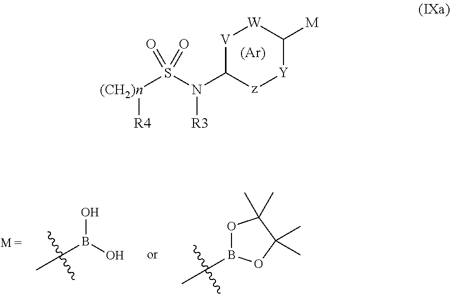
with a compound of formula (VII), in the presence of a coupling catalyst and a base, 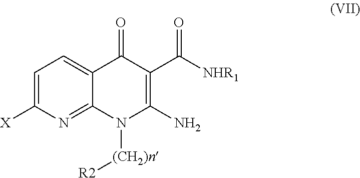
where R1, R2, R3, R4, n, n′, V, W, Y, Z and Ar are as defined in claim 1, X represents a leaving group and M is as defined above.

12. A process for preparing the compound according to claim 1, comprising reacting a compound of formula (IXb) 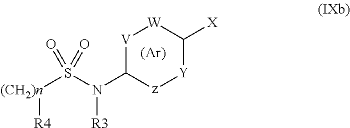
with a compound of formula (VIII), 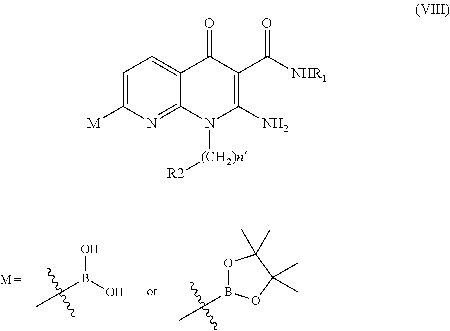
where R1, R2, R3, R4, n, n′, V, W, Y, Z and Ar are as defined in claim 1, X represents a leaving group and M is as defined above.

13. A compound of formula (VIII): 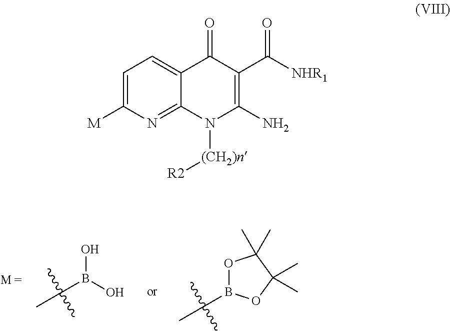
wherein:
n′ represents 0, 1, 2, 3 or 4;
R1 represents an alkyl group;
R2 represents:
(i) a cycloalkyl group,
(ii) an alkyl group, or
(iii) an alkoxy group,
said cycloalkyl, alkyl or alkoxy groups being optionally substituted with one or more halogen atoms; and
M is as defined above.

14. A pharmaceutical composition comprising the compound according to claim 1 or a pharmaceutically acceptable salt thereof, and at least one pharmaceutically acceptable excipient.

15. A method for treating a disease linked to the activity of protein kinases, comprising administering to a patient in need thereof a pharmaceutically effective amount of the compound according to claim 1 or a pharmaceutically acceptable salt thereof.

16. A method for treating a proliferative disease, comprising administering to a patient in need thereof a pharmaceutically effective amount of the compound according to claim 1 or a pharmaceutically acceptable salt thereof.

17. The method according to claim 17, wherein the proliferatuve disease is cancer, chronic or acute leukemia, lymphocytic lymphoma, Hodgkin's disease, myeloproliferative syndrome, or myelodysplastic syndrome.

18. A method for treating solid tumor cancer, lung cancer, bone cancer, pancreas cancer, skin cancer, Kaposi's syndrome, intraocular melanoma, breast cancer, uterus cancer, cervix cancer, ovarian cancer, endometrium cancer, vagina cancer, cancer of the vulva, of the urethra, penis cancer, prostate cancer, fallopian tube carcinoma, GISTs, cancer of the anal region, rectum cancer, small intestine cancer, colon cancer, stomach cancer, esophagus cancer, endocrine cancer, thyroid cancer, parathyroid or adrenal gland cancer, soft tissue sarcoma, Ewing's sarcoma, ostesarcoma, dermatofibrosarcoma or other fibrosarcoma, bladder cancer, kidney cancer, neoplasm of the central nervous system, vertebral column or desmoid tumor, brain stem glioma or glioblastoma, pituitary adenoma or metastasis thereof, chronic or acute leukemia, lymphocytic lymphoma, Hodgkin's disease, myeloproliferative syndrome, or myelodysplastic syndrome, comprising administering to a patient in need thereof a pharmaceutically effective amount of the compound according to claim 1 or a pharmaceutically acceptable salt thereof.

19. A method for treating a nonmalignant proliferative disease, restenosis, atherosclerosis, thrombosis, heart failure, cardiac hypertrophy, pulmonary arterial hypertension, fibrosis, diabetic nephropathy, glomerulonephritis, chronic pyelonephritis, hemangiomas, an autoimmune disease, psoriasis, sclerodermatitis, or immunosuppression, comprising administering to a patient in need thereof a pharmaceutically effective amount of the compound according to claim 1 or a pharmaceutically acceptable salt thereof.

20. A combination of at least one compound according to claim 1 with at least one chemotherapeutic agent.
